(19) United States
(12) Patent Application Publication
(10) Pub. No.: US 2022/0169779 A1
(43) Pub. Date: Jun. 2, 2022

(54) MATERIAL FOR MEDICAL USE AND METHOD FOR PRODUCING SAME

(71) Applicants: SHIN-ETSU CHEMICAL CO., LTD., Tokyo (JP); DAINICHISEIKA COLOR&CHEMICALS MFG. CO., LTD., Tokyo (JP); SHINSHU UNIVERSITY, NATIONAL UNIVERSITY CORPORATION, Matsumoto-shi, Nagano (JP)

(72) Inventors: Daisuke NODA, Annaka-shi (JP); Shinji IRIFUNE, Annaka-shi (JP); Masaki TANAKA, Tokyo (JP); Hiromasa SATO, Tokyo (JP); Motoaki UMEZU, Tokyo (JP); Shota IINO, Tokyo (JP); Toshihisa TANAKA, Ueda-shi (JP); Chuan Yin, Ueda-shi (JP)

(73) Assignees: SHIN-ETSU CHEMICAL CO., LTD., Tokyo (JP); DAINICHISEIKA COLOR&CHEMICALS MFG. CO., LTD., Tokyo (JP); SHINSHU UNIVERSITY, NATIONAL UNIVERSITY CORPORATION, Matsumoto-shi, Nagano (JP)

(21) Appl. No.: 17/434,647

(22) PCT Filed: Feb. 18, 2020
(86) PCT No.: PCT/JP2020/006190

§ 371 Date: Aug. 27, 2021

(30) Foreign Application Priority Data
Feb. 28, 2019 (JP) 2019-036533

(51) Int. Cl. C08G 18/38; C08G 18/76; C08G 18/32; A61L 27/18; D01D 5/00; D01F 6/70

(52) U.S. Cl. CPC C08G 18/3895; C08G 18/7692; D01F 6/70; A61L 27/18; D01D 5/003; C08G 18/3206

(57) ABSTRACT

A material for medical use, which contains nanofibers that are formed from a resin containing a silicone-modified polyurethane resin, and which is characterized in that: the silicone-modified polyurethane resin is a reaction product of (A) a long-chain polyol that has a number average molecular weight of 500 or more, (B) a short-chain polyol that has a number average molecular weight of less than 500, (C) an active hydrogen group-containing organopolysiloxane and (D) a polyisocyanate; and the average fiber diameter thereof is less than 2,000 nm. This material for medical use exhibits excellent performance in a biological test.

### TECHNICAL FIELD

This invention relates to a medical material comprising nanofibers containing a silicone-modified polyurethane resin. More particularly, it relates to a medical material comprising nanofibers which are formed from a resin containing a silicone-modified polyurethane resin and exhibit excellent properties in biological tests, preferably in the form of a fiber layup structure, and a method for preparing the same.

### BACKGROUND ART

Most resin fibers are obtained by a dry spinning method while resin fibers of certain type are obtained by a melt spinning method, wet spinning method or the like. Electrospinning methods (e.g., electrostatic spinning, electrospinning, and melt electrospinning methods) are known as methods for preparing fibers and fiber layup structures having a small fiber diameter which are difficult to manufacture by the above methods.

The electrospinning method is known as a method which is capable of forming extra-fine fibers by injecting a polymer-containing solution or a polymer melt under a high voltage, dividing the polymer into an extra-fine size due to electrostatic repulsion, and simultaneously solvent volatilization or cooling. Also, it is a fiber spinning method which is capable of yielding extra-fine fibers, a fiber layup structure, and non-woven fabric in one stage by collecting the resulting extra-fine fibers of the polymer. Typically, the fiber layup structure is formed via curing while the solvent is evaporated off from the polymer solution during the fiber spinning step. Curing may be performed by cooling (e.g., when the sample is liquid at high temperature), chemical curing (e.g., treating with curing vapor) or evaporating the solvent (e.g., when the sample is liquid at room temperature). The non-woven fabric is collected on a collecting substrate set in place, and may be detached therefrom if necessary.

Since nano-size fibers having a fiber diameter of less than 1,000 nm are known to have good super specific surface area effects, nano-size effects, and other effects as compared with ordinary fibers, active research and development works have been made on such nanofibers. In particular, the electrospinning method is widely used because nanofibers can be easily formed thereby.

Recently, applications of nanofibers to medical material (sometimes referred to as material for medical use) which take advantage of nanofiber characteristics have been proposed. Exemplary applications including cell scaffold materials, artificial organs, wound bandages, and tissue or bone regeneration membranes and also polyurethane nanofibers are described in JP-A 2016-055195, JP-A 2017-064311, JP-A 2015-198604, and WO 2007/063820 (Patent Documents 1 to 4).

Fibers and fiber layup structures based on silicone-modified polyurethane resins are disclosed in WO 2016/158967 and WO 2017/175680 (Patent Documents 5 and 6). They have excellent properties including flexibility, slippage, blocking resistance, heat retention, water vapor permeability, water repellency, and spinnability as compared with fibers of polyurethane resins and nanofibers of silicone resins.

Although fibers based on silicone-modified polyurethane resins have excellent properties over the polyurethane nanofibers as mentioned above, they have not been evaluated by biological tests and the effect of silicone modification is unclear. It is thus indefinite whether or not they are applicable as the medical material.

### PRIOR ART DOCUMENTS

### Patent Documents

Patent Document 1: JP-A 2016-055195

Patent Document 2: JP-A 2017-064311

Patent Document 3: JP-A 2015-198604

Patent Document 4: WO 2007/063820

Patent Document 5: WO 2016/158967

Patent Document 6: WO 2017/175680

### SUMMARY OF INVENTION

### Technical Problem

An object of the invention, which has been made under the above-mentioned circumstances, is to provide a medical material comprising nanofibers which exhibit excellent properties in biological tests.

### Solution to Problem

Making extensive investigations to attain the above object, the inventors have found that nanofibers formed from a resin containing a silicone-modified polyurethane resin exhibit excellent properties in biological tests as compared with non-silicone-modified polyurethane resins. The invention is predicated on this finding.

The invention provides a medical material and a method for preparing the same, as defined below.

1.

A medical material comprising nanofibers formed from a resin containing a silicone-modified polyurethane resin, the silicone-modified polyurethane resin comprising the reaction product of (A) a long chain polyol having a number average molecular weight of at least 500, (B) a short chain polyol having a number average molecular weight of less than 500, (C) an active hydrogen-containing organopolysiloxane, and (D) a polyisocyanate, the nanofibers having an average fiber diameter of less than 2,000 nm.

2.

The medical material of 1 wherein component (C) is (C-1) an organopolysiloxane represented by the following formula (1) and/or (C-2) an organopolysiloxane having carbinol groups only at one end of the molecular chain, represented by the following formula (2):

R¹R²₂SiO(SiR²₂O)ₘSiR¹R²₂ (1)

wherein R¹ is each independently a C₁-C₁₀ monovalent hydrocarbon group which has a hydroxy or mercapto group and may have an oxygen atom intervening in the chain, or a C₁-C₁₀ monovalent hydrocarbon group having a primary or secondary amino group, R² is each independently a straight, branched or cyclic C₁-C₁₀ alkyl or aralkyl group in which some hydrogen may be substituted by fluorine, optionally substituted C₅-C₁₂ aryl group, or vinyl group, and m is an integer of 1 to 200,

R²₃SiO(SiR²₂O)ₙSiR²₂R⁴ (2)

wherein R² is as defined above, R⁴ is a characteristic group having the following formula (3):

—R⁵—X—CH₂C(R⁶)₂R⁷ (3)

wherein R⁵ is a C₂-C₁₀ alkylene group which may contain an oxygen atom in the chain, R⁶ is a C₁-C₁₀ carbinol group, R⁷ is hydrogen, an amino group or C₁-C₁₀ alkyl group, and X is a single bond or —O— bond, with the proviso that when R⁵ contains an oxygen atom and X is a —O— bond, these two oxygen atoms do not adjoin each other, and n is an integer of 1 to 200.
3.

The medical material of 1 or 2 wherein the average fiber diameter is less than 1,000 nm.

4.

The medical material of any one of 1 to 3 wherein component (C) is blended in an amount of 0.1 to 50 parts by weight per 100 parts by weight of components (A) to (D) combined.

5.

The medical material of any one of 1 to 4, which is in the form of a fiber layup structure comprising the nanofibers.

6.

The medical material of any one of 1 to 5, which is a cell scaffold material.

7.

The medical material of any one of 1 to 5, which is a wound dressing material.

8.

A method for preparing the nanofiber-containing medical material of any one of 1 to 7, comprising the step of spinning the nanofibers from a solution or dispersion of the silicone-modified polyurethane resin by an electrospinning method.

9.

The method of 8, further comprising the step of washing and/or drying the nanofibers spun by an electrospinning method.

10.

The method of 9, further comprising the step of sterilizing the washed and/or dried nanofibers.

### Advantageous Effects of Invention

The inventive medical material has the characteristics of nanofibers and fiber layup structures thereof, shows better cell adhesion and cell proliferation than similar nanofibers of non-silicone-modified polyurethane resins, and possesses biocompatibility. The medical material is thus quite useful as cell scaffold materials and wound dressing materials.

### BRIEF DESCRIPTION OF DRAWINGS

FIG. 1 is a schematic view for illustrating one exemplary setup in which a non-woven fabric is prepared by injecting a polymer solution in an electrostatic field using an electrospinning (i.e., electrostatic spinning) method.

FIG. 2 is an image showing the SEM observation result after 3-day cell incubation using structure 1-1 in Example 2-1 in cell proliferation assay.

FIG. 3 is an image showing the SEM observation result after 3-day cell incubation using structure 1-2 in Example 2-2 in cell proliferation assay.

FIG. 4 is an image showing the SEM observation result after 3-day cell incubation using structure 1-3 in Example 2-3 in cell proliferation assay.

FIG. 5 is an image showing the SEM observation result after 3-day cell incubation using structure 4 in Example 2-4 in cell proliferation assay.

FIG. 6 is an image showing the SEM observation result after 3-day cell incubation using structure 5 in Example 2-5 in cell proliferation assay.

FIG. 7 is an image showing the SEM observation result after 3-day cell incubation using structure 7 in Example 2-6 in cell proliferation assay.

FIG. 8 is an image showing the SEM observation result after 3-day cell incubation using structure 8 in Example 2-7 in cell proliferation assay.

FIG. 9 is an image showing the SEM observation result after 3-day cell incubation using comparative structure 1 in Comparative Example 2-1 in cell proliferation assay.

FIG. 10 is a micrograph showing the microscopic observation result of structure 4 (SiPU4) after cytotoxicity evaluation in Example 3.

FIG. 11 is a micrograph showing the microscopic observation result of structure 8 (SiPU8) after cytotoxicity evaluation in Example 3.

FIG. 12 is a micrograph showing the microscopic observation result of a positive control material after cytotoxicity evaluation in Example 3.

### DESCRIPTION OF EMBODIMENTS

### [Medical Material]

Now the invention is described in detail.

The invention provides a medical material comprising nanofibers formed from a resin containing a silicone-modified polyurethane resin, the silicone-modified polyurethane resin being the reaction product of components (A), (B), (C), and (D), and the nanofibers having an average fiber diameter of less than 2,000 nm.

### (Silicone-Modified Polyurethane Resin)

The silicone-modified polyurethane resin is obtained from reaction of (A) a long chain polyol having a number average molecular weight of at least 500, (B) a short chain polyol having a number average molecular weight of less than 500, (C) an active hydrogen-containing organopolysiloxane, and (D) a polyisocyanate. The active hydrogen-containing organopolysiloxane (C) is preferably present in an amount of 0.1 to 50 parts, more preferably 0.1 to 40 parts, and even more preferably 1 to 30 parts by weight per 100 parts by weight of components (A) to (D) combined.

As used herein, the term “reaction product” refers to not only a reaction product consisting of components (A) to (D), but also a reaction product including components (A) to (D) and another component such as (E) a polyamine. The term “active hydrogen (group)” refers to a hydrogen atom in alcohols, amines, thiols, and the like.

Herein, the silicone-modified polyurethane resin may be prepared using a well-known polyurethane synthesis method.

### ((A) Long Chain Polyol Having a Number Average Molecular Weight of at Least 500)

Component (A) is a high molecular weight polyol having a number average molecular weight (Mn) of at least 500, preferably 500 to 10,000, and more preferably 700 to 3,000, excluding (C) the active hydrogen-containing organopolysiloxane. Examples of the high molecular weight polyol include those belonging to groups (i) to (vi) described below. It is noted that the number average molecular weight used herein is determined by gel permeation chromatography (GPC) versus polymethyl methacrylate (PMMA) standards using tetrahydrofuran (THF) as developing solvent (the same holds true, hereinafter).

(i) Polyether polyols;

for example, those obtained from polymerization or copolymerization of an alkylene oxide (e.g., ethylene oxide, propylene oxide or butylene oxide) and/or a cyclic ether (e.g., tetrahydrofuran), such as polyethylene glycol, polypropylene glycol, polyethylene glycol-polytetramethylene glycol (block or random copolymer), poly(tetramethylene ether) glycol, and polyhexamethylene glycol

(ii) Polyester polyols;

for example, those obtained from condensation polymerization of an aliphatic dicarboxylic acid (e.g., succinic acid, adipic acid, sebacic acid, glutaric acid or azelaic acid) and/or an aromatic dicarboxylic acid (e.g., isophthalic acid or terephthalic acid) with a low-molecular-weight glycol (e.g., ethylene glycol, 1,2-propylene glycol, 1,3-propylene glycol, 1,4-butylene glycol, 1,6-hexamethylene glycol, neopentyl glycol or 1,4-bishydroxymethylcyclohexane), such as poly(ethylene adipate) diol, poly(butylene adipate) diol, poly(hexamethylene adipate) diol, poly(neopentyl adipate) diol, poly(ethylene/butylene adipate) diol, poly(neopentyl/hexyl adipate) diol, poly(3-methylpentane adipate) diol, and poly(butylene isophthalate) diol

(iii) Polylactone polyols;

for example, polycaprolactone diol or triol and poly-3-methylvalerolactone diol

(iv) Polycarbonate polyols;

for example, poly(trimethylene carbonate) diol, poly(tetramethylene carbonate) diol, poly(pentamethylene carbonate) diol, poly(neopentyl carbonate) diol, poly(hexamethylene carbonate) diol, poly(1,4-cyclohexanedimethylene carbonate) diol, poly(decamethylene carbonate) diol, and random/block copolymers thereof

(v) Polyolefin polyols;

for example, polybutadiene glycol, polyisoprene glycol, and hydrides thereof

(vi) Polymethacrylate polyols;

for example, α,ω-polymethyl methacrylate diol and α,ω-polybutyl methacrylate diol

Of these, polyether polyols are preferred, with polyethylene glycol, polypropylene glycol, and poly(tetramethylene ether) glycol being more preferred.

((B) Short Chain Polyol Having a Number Average Molecular Weight of Less than 500)

Component (B) is a short chain polyol having a number average molecular weight (Mn) of less than 500, preferably 60 to less than 500, and more preferably 60 to 300, examples of which include aliphatic glycols such as ethylene glycol, 1,2-propanediol, 1,3-propanediol, 1,4-butanediol, 1,3-butanediol, 1,6-hexamethylene glycol, and neopentyl glycol, and alkylene oxide low mole adducts thereof (Mn of less than 500); alicyclic glycols such as 1,4-bishydroxymethylcyclohexane and 2-methyl-1,1-cyclohexanedimethanol, and alkylene oxide low mole adducts thereof (Mn of less than 500); aromatic glycols such as xylylene glycol, and alkylene oxide low mole adducts thereof (Mn of less than 500); bisphenols such as bisphenol A, thiobisphenol, and sulfonebisphenol, and alkylene oxide low mole adducts thereof (Mn of less than 500); alkyl dialkanol amines such as C₁-C₁₈ alkyl diethanol amines; and polyhydric alcohol compounds such as glycerol, trimethylolethane, trimethylolpropane, pentaerythritol, tris(2-hydroxyethyl)isocyanurate, 1,1,1-trimethylolethane, and 1,1,1-trimethylolpropane. Of these, aliphatic glycols are preferred, with ethylene glycol, 1,3-propanediol, and 1,4-butanediol being more preferred.

Component (B) is preferably blended in an amount of 1 to 200 parts, more preferably 10 to 30 parts by weight per 100 parts by weight of the long chain polyol (A).

### ((C) Active Hydrogen-Containing Organopolysiloxane)

Component (C) should preferably be (C-1) an organopolysiloxane represented by the formula (1) and/or (C-2) an organopolysiloxane having carbinol groups only at one end of the molecular chain, represented by the formula (2).

### ((C-1) Organopolysiloxane)

Component (C-1) is an organopolysiloxane represented by the following formula (1):

R¹R²₂SiO(SiR²₂O)ₘSiR¹R²₂ (1)

wherein IV is each independently a C₁-C₁₀ monovalent hydrocarbon group which has a hydroxy or mercapto group and may have an oxygen atom intervening in the chain, or a C₁-C₁₀ monovalent hydrocarbon group having a primary or secondary amino group, R² is each independently a straight, branched or cyclic C₁-C₁₀ alkyl or aralkyl group in which some hydrogen may be substituted by fluorine, optionally substituted C₅-C₁₂ aryl group, or vinyl group, and m is an integer of 1 to 200.

Examples of the C₁-C₁₀ monovalent hydrocarbon group which has a hydroxy or mercapto group and may have an oxygen atom intervening in the chain, represented by R¹, include hydroxymethyl, 2-hydroxyethan-1-yl, 2-hydroxypropan-1-yl, 3-hydroxypropan-1-yl, 2-hydroxybutan-1-yl, 3-hydroxybutan-1-yl, 4-hydroxybutan-1-yl, 2-hydroxyphenyl, 3-hydroxyphenyl, 4-hydroxyphenyl, 2-(hydroxymethoxy)ethan-1-yl, 2-(2-hydroxyethoxy)ethan-1-yl, 2-(2-hydroxypropoxy)ethan-1-yl, 2-(3-hydroxypropoxy)ethan-1-yl, 2-(2-hydroxybutoxy)ethan-1-yl, 2-(3-hydroxybutoxy)ethan-1-yl, 2-(4-hydroxybutoxy)ethan-1-yl, 3-(hydroxymethoxy)propan-1-yl, 3-(2-hydroxyethoxy)propan-1-yl, 3-(2-hydroxypropoxy)propan-1-yl, 3-(3-hydroxypropoxy)propan-1-yl, 3-(2-hydroxybutoxy)propan-1-yl, 3-(3-hydroxybutoxy)propan-1-yl, 3-(4-hydroxybutoxy)propan-1-yl, mercaptomethyl, 2-mercaptoethan-1-yl, 2-mercaptopropan-1-yl, 3-mercaptopropan-1-yl, 2-mercaptobutan-1-yl, 3-mercaptobutan-1-yl, 4-mercaptobutan-1-yl, 2-(mercaptomethoxy)ethan-1-yl, 2-(2-mercaptoethoxy)ethan-1-yl, 2-(2-mercaptopropoxy)ethan-1-yl, 2-(3-mercaptopropoxy)ethan-1-yl, 2-(2-mercaptobutoxy)ethan-1-yl, 2-(3-mercaptobutoxy)ethan-1-yl, 2-(4-mercaptobutoxy)ethan-1-yl, 3-(mercaptomethoxy)propan-1-yl, 3-(2-mercaptoethoxy)propan-1-yl, 3-(2-mercaptopropoxy)propan-1-yl, 3-(3-mercaptopropoxy)propan-1-yl, 3-(2-mercaptobutoxy)propan-1-yl, 3-(3-mercaptobutoxy)propan-1-yl, and 3-(4-mercaptobutoxy)propan-1-yl.

Examples of the C₁-C₁₀ monovalent hydrocarbon group having a primary or secondary amino group, represented by R¹, include aminomethyl, 2-aminoethan-1-yl, 2-aminopropan-1-yl, 3-aminopropan-1-yl, 2-aminobutan-1-yl, 3-aminobutan-1-yl, 4-aminobutan-1-yl, N-methylaminomethyl, N-methyl-2-aminoethan-1-yl, N-methyl-2-aminopropan-1-yl, N-methyl-3-aminopropan-1-yl, N-methyl-2-aminobutan-1-yl, N-methyl-3-aminobutan-1-yl, N-methyl-4-aminobutan-1-yl, N-ethylaminomethyl, N-ethyl-2-aminoethan-1-yl, N-ethyl-2-aminopropan-1-yl, N-ethyl-3-aminopropan-1-yl, N-ethyl-2-aminobutan-1-yl, N-ethyl-3-aminobutan-1-yl, N-ethyl-4-aminobutan-1-yl, N-butylaminomethyl, N-butyl-2-aminoethan-1-yl, N-butyl-2-aminopropan-1-yl, N-butyl-3-aminopropan-1-yl, N-butyl-2-aminobutan-1-yl, N-butyl-3-aminobutan-1-yl, and N-butyl-4-aminobutan-1-yl.

Of these, IV is preferably a C₂-C₆ monovalent hydrocarbon group having a primary or secondary hydroxy group and optionally having an oxygen atom intervening in the chain or a C₂-C₆ monovalent hydrocarbon group having a primary or secondary amino group, more preferably 2-hydroxyethan-1-yl, 3-hydroxypropan-1-yl, 3-(2-hydroxyethoxy)propan-1-yl or 3-aminopropan-1-yl.

Examples of the straight, branched or cyclic C₁-C₁₀ alkyl or aralkyl group R² include methyl, ethyl, propyl, isopropyl, n-butyl, cyclohexyl, 2-ethylhexan-1-yl, 2-phenylethan-1-yl, and 2-methyl-2-phenylethan-1-yl.

Examples of the straight, branched or cyclic C₁-C₁₀ alkyl group in which some hydrogen is substituted by fluorine, represented by R², include 3,3,3-trifluoropropyl, 3,3,4,4,4-pentafluorobutyl, 3,3,4,4,5,5,6,6,6-nonafluorohexyl, 3,3,4,4,5,5,6,6,7,7,7-undecafluoroheptyl, 3,3,4,4,5,5,6,6,7,7,8,8,8-tridecafluorooctyl, 3,3,4,4,5,5,6,6,7,7,8,8,9,9,9-pentadecafluorononyl, and 3,3,4,4,5,5,6,6,7,7,8,8,9,9,10,10,10-heptadecafluorodecyl.

Examples of the optionally substituted C₅-C₁₂ aryl group R² include phenyl, 2-methyl-1-phenyl, 3-methyl-1-phenyl, 4-methyl-1-phenyl, 2,3-dimethyl-1-phenyl, 3,4-dimethyl-1-phenyl, 2,3,4-trimethyl-1-phenyl, 2,4,6-trimethyl-1-phenyl, and naphthyl.

Of these, R² is preferably selected from the group consisting of methyl, phenyl, 3,3,3-trifluoropropyl, and vinyl.

In formula (1), m is an integer of 1 to 200, preferably an integer of 1 to 40, and most preferably an integer of 5 to 30. If m is greater than the range, a viscosity buildup and a decline of terminal reactivity of silicone moiety and degradation of urethane resin properties are observable.

The organopolysiloxane as component (C-1) may be synthesized in a way corresponding to a desired substituent while a commercially available one may be used. Examples include the following groups of Compounds (1-1) to (4-7). In the following formulae, Me stands for methyl and Ph for phenyl (the same applies hereinafter).

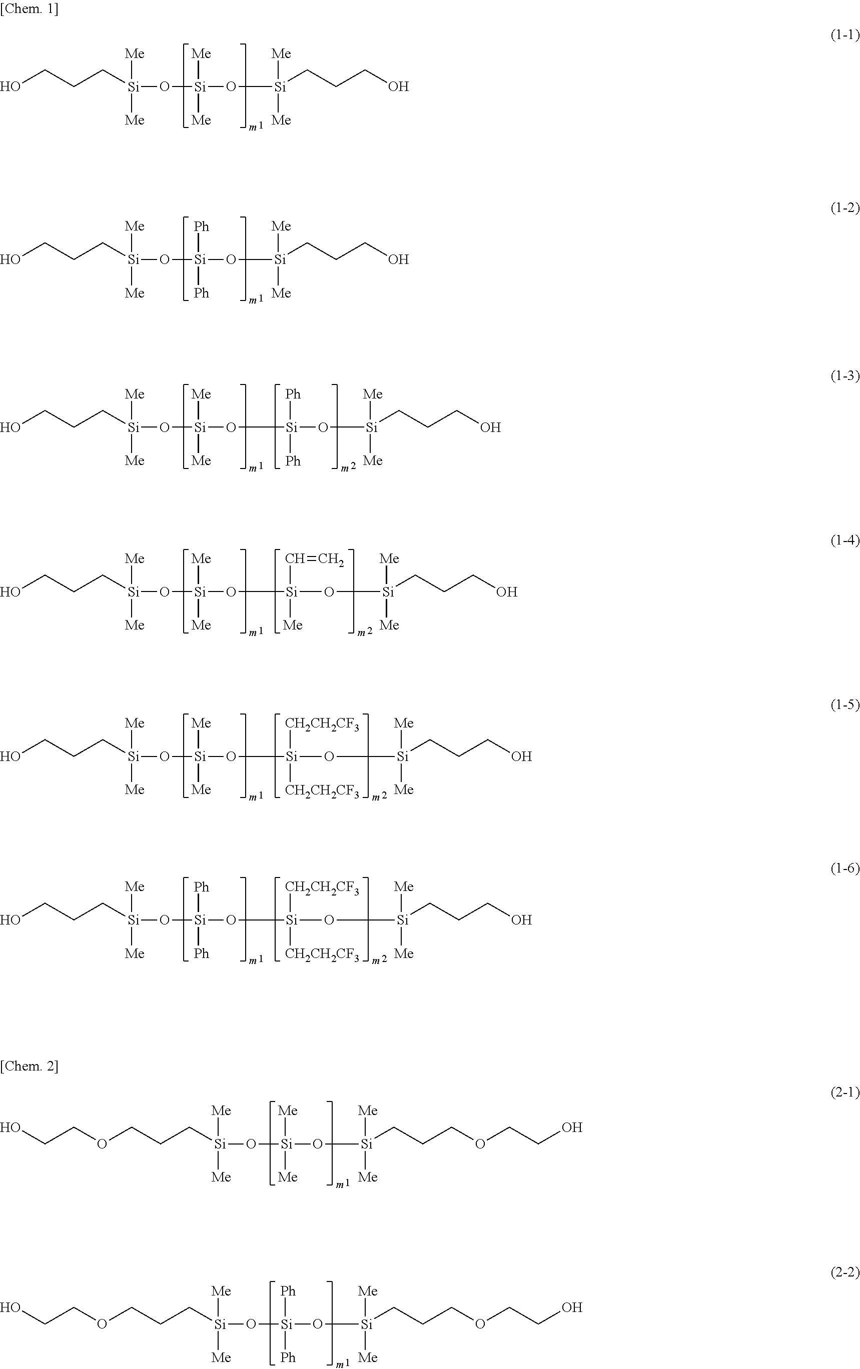

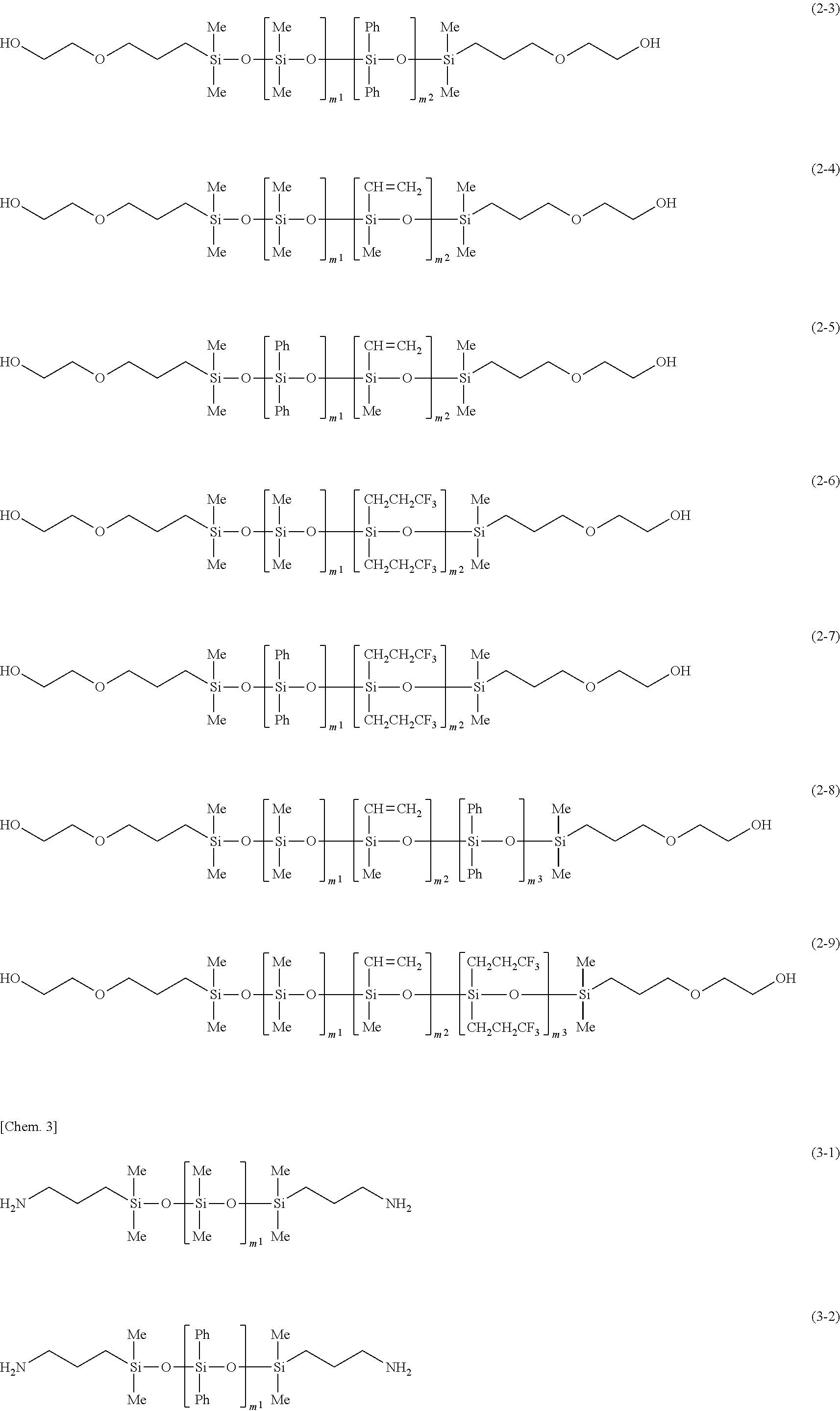

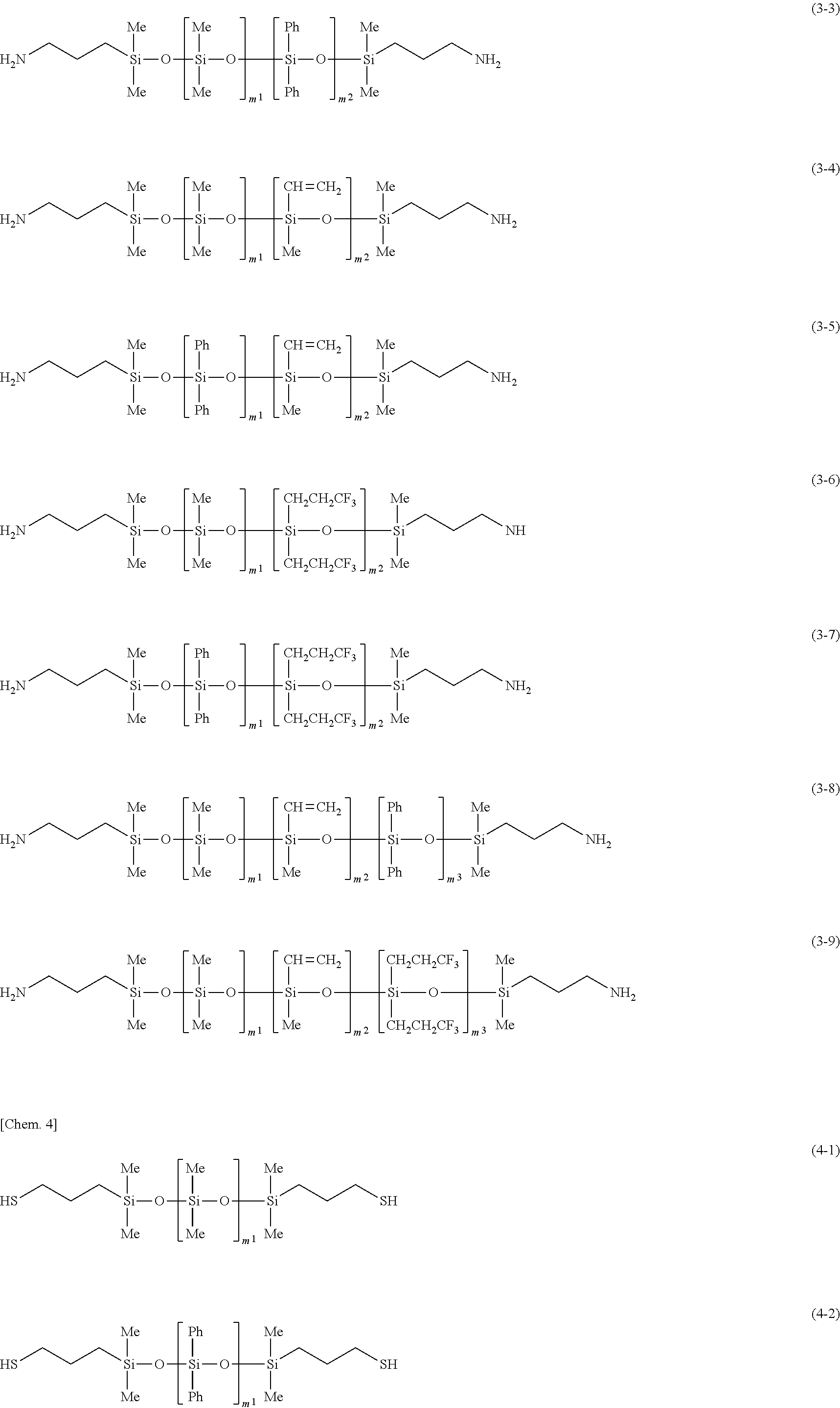

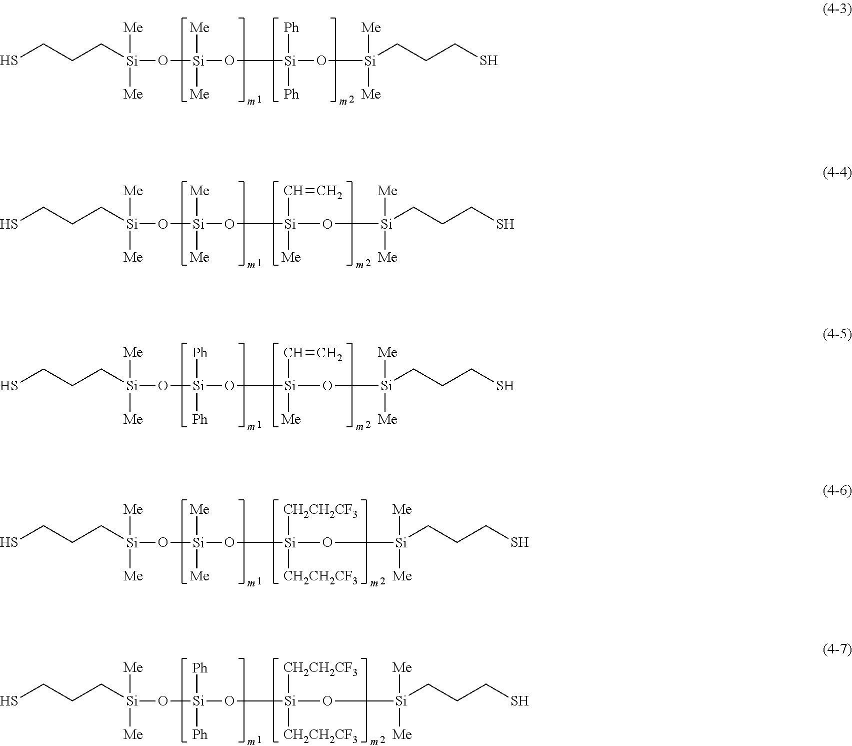

Notably, in the groups of Compounds (1-1), (1-2), (2-1), (2-2), (3-1), (3-2), (4-1) and (4-2), m¹=m wherein m¹ is at least 1; in the groups of Compounds (1-3) to (1-6), (2-3) to (2-7), (3-3) to (3-7), and (4-3) to (4-7), m¹+m²=m wherein m¹ is at least 1, m² is at least 1, and m is an integer of 2 to 200; and in the groups of Compounds (2-8), (2-9), (3-8), and (3-9), m¹+m²+m³=m wherein m¹, m², and m³ each are at least 1 and m is an integer of 3 to 200.

These compounds may be synthesized by reacting an active hydrogen-containing disiloxane with a cyclotetrasiloxane having an arbitrary substituent under acidic or alkaline conditions.

### ((C-2) Organopolysiloxane Having Carbinol Groups Only at One End of the Molecular Chain)

Component (C-2) is an organopolysiloxane having two carbinol groups only at one end of the molecular chain, represented by the following formula (2):

R²₃SiO(SiR²₂O)ₙSiR²₂R⁴ (2)

wherein R² is as defined above, R⁴ is a characteristic group having the following formula (3):

—R⁵—X—CH₂C(R⁶)₂R⁷ (3)

wherein R⁵ is a C₂-C₁₀ divalent alkylene group which may contain an oxygen atom in the chain, R⁶ is a C₁-C₁₀ carbinol group, R⁷ is hydrogen, an amino group or C₁-C₁₀ monovalent alkyl group, and X is a single bond or —O— bond, with the proviso that when R⁵ contains an oxygen atom and X is a —O— bond, these two oxygen atoms do not adjoin each other, and n is an integer of 1 to 200.

In formula (2), R² is as defined above, that is, it is the same as R² in formula (1).

In formula (3) representative of R⁴, R⁵ is a C₂-C₁₀ alkylene group optionally containing an oxygen atom in the chain, which is selected from, for example, 1,2-ethylene, 1,2-propylene, 1,3-propylene, 1,3-butylene, 1,4-butylene, 1,3-pentylene, 1,4-pentylene, 1,5-pentylene, 1,6-hexylene, 1,7-heptylene, 1,8-octylene, 1,9-nonylene, 1,10-decylene, 2-(3-propan-1-oxy)eth-1-ylene, 3-(3-propan-1-oxy)prop-1-ylene, 4-(3-propan-1-oxy)but-1-ylene, 5-(3-propan-1-oxy)pent-1-ylene, 6-(3-propan-1-oxy)hex-1-ylene, 1,3-cyclohexylene, 1,4-cyclohexylene, 1,3-cycloheptylene, 1,4-cycloheptylene, and 1,4-dioxacyclohex-2,5-ylene. For availability, 1,3-propylene is preferred.

In formula (3), R⁶ is a C₁-C₁₀ carbinol group, examples of which include hydroxymethyl, 2-hydroxyeth-1-yl, 2-hydroxyprop-1-yl, 3-hydroxyprop-1-yl, 2-hydroxybut-1-yl, 4-hydroxybut-1-yl, 5-hydroxypent-1-yl, 6-hydroxyhex-1-yl, 7-hydroxyhept-1-yl, 8-hydroxyoct-1-yl, 9-hydroxynon-1-yl, and 10-hydroxydec-1-yl. Of these, hydroxymethyl and 2-hydroxyeth-1-yl are preferred.

In formula (3), R⁷ is hydrogen or a C₁-C₁₀ alkyl group, examples of which include methyl, ethyl, propyl, isopropyl, n-butyl, cyclohexyl, 2-ethylhexan-1-yl, 2-phenylethan-1-yl, and 2-methyl-2-phenylethan-1-yl. Of these, hydrogen, methyl, and ethyl are preferred.

In formula (3), X is a single bond or —O— bond.

In formula (2), n is an integer of 1 to 200, preferably an integer of 10 to 160. If n is greater than the range, a viscosity buildup and a decline of terminal reactivity of silicone moiety and degradation of urethane resin properties are observable.

The organopolysiloxane as component (C-2) may be synthesized in a way corresponding to a desired substituent. Examples include the following Compounds (5-1) to (5-6). In the following formulae, Bu stands for butyl (the same applies hereinafter).

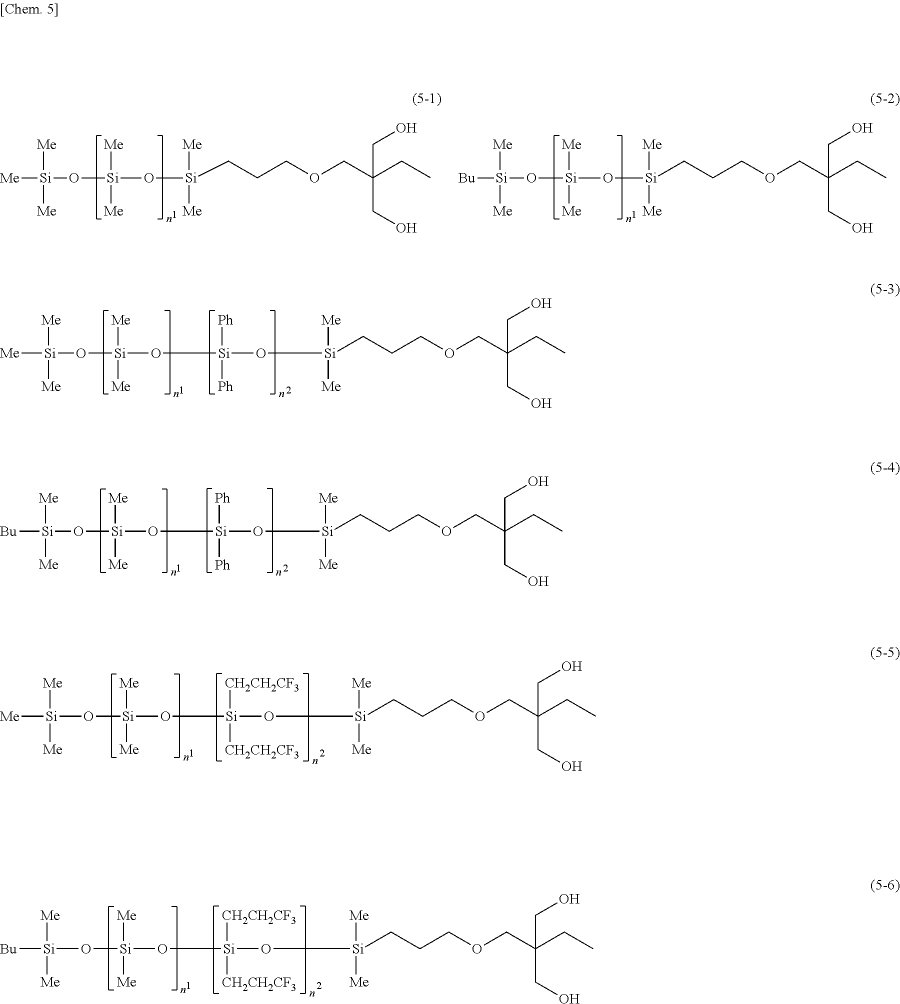

Notably, in Compounds (5-1) and (5-2), n¹=n wherein n¹ is at least 1. In Compounds (5-3) to (5-6), n¹+n²=n wherein n¹ is at least 1, n² is also at least 1, and n is an integer of 2 to 200.

These compounds may be synthesized by effecting hydrosilylation reaction of one end hydrogen polydimethylsiloxane with trimethylolpropane monoallyl ether.

Although the active hydrogen-containing organopolysiloxane as component (C) may consist of the organopolysiloxane as component (C-1) or the organopolysiloxane having carbinol groups only at one end of the molecular chain as component (C-2), it may be a mixture of components (C-1) and (C-2).

The weight ratio of component (C-1) to component (C-2) is preferably from 100:0 to 0:100. When component (C-2) is blended, the ratio is preferably from 99:1 to 1:99.

### ((D) Polyisocyanate)

Component (D) used herein may be any of well-known polyisocyanates. Preferred examples include aromatic diisocyanates such as toluene-2,4-diisocyanate, 4-methoxy-1,3-phenylene diisocyanate, 4-isopropyl-1,3-phenylene diisocyanate, 4-chloro-1,3-phenylene diisocyanate, 4-butoxy-1,3-phenylene diisocyanate, 2,4-diisocyanatodiphenyl ether, 4,4′-methylenebis(phenylene isocyanate) (MDI), durylene diisocyanate, tolidine diisocyanate, xylylene diisocyanate (XDI), 1,5-naphthalene diisocyanate, benzidine diisocyanate, o-nitrobenzidine diisocyanate, and 4,4′-diisocyanate dibenzyl; aliphatic diisocyanates such as methylene diisocyanate, 1,4-tetramethylene diisocyanate, 1,6-hexamethylene diisocyanate, and 1,10-decamethylene diisocyanate; alicyclic diisocyanates such as 1,4-cyclohexylene diisocyanate, 1,5-tetrahydronaphthalene diisocyanate, isophorone diisocyanate, 4,4′-methylenebis(cyclohexyl isocyanate) (H12MDI), and hydrogenated XDI; and polyurethane prepolymers obtained by reacting these diisocyanate compounds with low-molecular-weight polyols or polyamines so as to be terminated with isocyanate. Of these, 4,4′-methylenebis(phenylene isocyanate), xylylene diisocyanate, 1,6-hexamethylene diisocyanate, isophorone diisocyanate, 4,4′-methylenebis(cyclohexyl isocyanate) (H12MDI), and hydrogenated XDI are more preferred.

Component (D) is blended in such an amount that the equivalent ratio of isocyanate groups to active hydrogen groups derived from components (A) to (C) may fall in a range of preferably from 0.9 to 1.1, more preferably from 0.95 to 1.05, and even more preferably from 0.99 to 1.01. An equivalent ratio within the range is preferred because physical properties of urethane are not compromised and fibers can be spun in a stable manner and endowed with strength.

### (Other Components)

### ((E) Polyamine)

A polyamine may be added as component (E) in the synthesis of the silicone-modified polyurethane resin used herein. Examples of the polyamine (E) include short chain diamines (e.g., aliphatic diamine compounds, aromatic diamine compounds, and alicyclic diamine compounds), long chain diamines, and hydrazines, excluding the active hydrogen-containing organopolysiloxane as component (C). Exemplary short chain diamines include aliphatic diamine compounds such as ethylenediamine, trimethylenediamine, hexamethylenediamine, trimethylhexamethylenediamine, and octamethylenediamine, aromatic diamine compounds such as phenylenediamine, 3,3′-dichloro-4,4′-diaminodiphenylmethane, 4,4′-methylenebis(phenylamine), 4,4′-diaminodiphenyl ether, and 4,4′-diaminodiphenyl sulfone, and alicyclic diamine compounds such as cyclopentanediamine, cyclohexyldiamine, 4,4-diaminodicyclohexylmethane, 1,4-diaminocyclohexane, and isophoronediamine. Exemplary long chain diamines include those obtained from polymerization or copolymerization of an alkylene oxide (e.g., ethylene oxide, propylene oxide or butylene oxide), such as polyoxyethylenediamine and polyoxypropylenediamine. Exemplary hydrazines include hydrazine, carbodihydrazide, adipic acid dihydrazide, sebacic acid dihydrazide, and phthalic acid dihydrazide.

When amino-modified silane coupling agents are used, it is possible to design self-curing reaction type paints. Examples include N-2-(aminoethyl)-3-aminopropylmethyldimethoxysilane (KBM-602, Shin-Etsu Chemical Co., Ltd.), N-2-(aminoethyl)-3-aminopropylmethyltrimethoxysilane (KBM-603, Shin-Etsu Chemical Co., Ltd.), N-2-(aminoethyl)-3-aminopropylmethyldiethoxysilane (KBE-602, Shin-Etsu Chemical Co., Ltd.), 3-aminopropyltrimethoxysilane (KBE-603, Shin-Etsu Chemical Co., Ltd.), 3-aminopropyltriethoxysilane (KBE-903, Shin-Etsu Chemical Co., Ltd.), and 3-ureidopropyltriethoxysilane.

When used, the polyamine (E) is preferably blended in an amount of 1 to 30 parts, more preferably 1 to 15 parts by weight per 100 parts by weight of components (A) to (D) combined.

### (Catalyst)

If necessary, a catalyst may be used in the synthesis of the silicone-modified polyurethane resin used herein. Examples of the catalyst include dibutyltin dilaurate, dibutyltin maleate, dibutyltin phthalate, dibutyltin dioctanoate, dibutyltin bis(2-ethylhexanoate), dibutyltin bis(methylmaleate), dibutyltin bis(ethylmaleate), dibutyltin bis(butylmaleate), dibutyltin bis(octylmaleate), dibutyltin bis(tridecylmaleate), dibutyltin bis(benzylmaleate), dibutyltin diacetate, dibutyltin bisisooctylthioglycolate, dibutyltin bis-2-ethylhexylthioglycolate, dioctyltin bis(ethylmaleate), dioctyltin bis(octylmaleate), dibutyltin dimethoxide, dibutyltin bis(nonylphenoxide), dibutenyltin oxide, dibutyltin oxide, dibutyltin bis(acetylacetonate), dibutyltin bis(ethylacetoacetonate), the reaction product of dibutyltin oxide with a silicate compound, the reaction product of dibutyltin oxide and phthalate, lead octoate, tetrabutyl titanate, tetrapropyl titanate, tetraisopropyl titanate, titanium tetrakis(acetylacetonate), titanium diisopropoxy bis(acetylacetonate), titanium diisopropoxy bis(ethylacetate), salts of metals with organic or inorganic acids, such as complexes obtained by reaction of titanium chloride or the like with a diol such as tartaric acid, organometal derivatives, and tertiary organic base catalysts such as trimethylamine, triethylamine (Et₃N), diisopropylethylamine (DIPEA), tri-n-butylamine, tri-n-pentylamine, tri-n-hexylamine, tri-n-heptylamine, tri-n-octylamine, N-methylpyrrolidine, N-methylpiperidine, N-methylmorpholine (NMO), N,N,N′,N′-tetramethylethylenediamine (TMEDA), N-methylimidazole (NMI), pyridine, 2,6-lutidine, 1,3,5-collidine, N,N-dimethylaminopyridine (DMAP), pyrazine, quinoline, 1,8-diazabicyclo[5,4,0]-7-undecene (DBU), and 1,4-diazabicyclo[2,2,2]octane (DABCO).

The catalyst is blended in a catalytic amount, preferably 0.01 to 10% by weight, more preferably 0.1 to 5% by weight based on the total amount of components (A) to (E).

### (Organic Solvent)

The silicone-modified polyurethane resin used herein may be synthesized in a solventless system or in an organic solvent if necessary. The preferred organic solvents include solvents which are inert to isocyanate groups or have lower activity than active hydrogen groups (alcohols, amines or thiols) in components (A) to (E). Examples include ketone solvents, e.g., acetone, methyl ethyl ketone, methyl isobutyl ketone, cyclohexanone, and menthone; aromatic hydrocarbon solvents, e.g., toluene, o-xylene, m-xylene, p-xylene, 1,3,5-mesitylene, 1,2,3-mesitylene, 1,2,4-mesitylene, ethylbenzene, n-propylbenzene, i-propylbenzene, n-butylbenzene, i-butylbenzene, sec-butylbenzene, t-butylbenzene, n-pentylbenzene, i-pentylbenzene, sec-pentylbenzene, t-pentylbenzene, n-hexylbenzene, i-hexylbenzene, sec-hexylbenzene, t-hexylbenzene, Swazole (aromatic hydrocarbon solvent from Cosmo Oil Co., Ltd.), and Solvesso (aromatic hydrocarbon solvent from Exxon Chemical Co., Ltd.); aliphatic hydrocarbon solvents, e.g., pentane, hexane, heptane, octane, nonane, cyclopentane, cyclohexane, cycloheptane, cyclooctane, methylcyclohexane, ethylcyclohexane, propylcyclohexane, n-butylcyclohexane, i-butylcyclohexane, sec-butylcyclohexane, t-butylcyclohexane, n-pentylcyclohexane, i-pentylcyclohexane, sec-pentylcyclohexane, t-pentylcyclohexane, n-hexylcyclohexane, i-hexylcyclohexane, sec-hexylcyclohexane, t-hexylcyclohexane, and limonene; alcohol solvents, e.g., methyl alcohol, ethyl alcohol, isopropyl alcohol, s-butyl alcohol, isobutyl alcohol, and t-butyl alcohol; ether solvents, e.g., diethyl ether, t-butyl methyl ether (TBME), dibutyl ether, cyclopentyl methyl ether (CPME), diphenyl ether, dimethoxymethane (DMM), tetrahydrofuran (THF), 2-methyltetrahydrofuran, 2-ethyltetrahydrofuran, tetrahydropyran (THP), dioxane, trioxane, 1,2-dimethoxyethane, diethylene glycol dimethyl ether, and diethylene glycol diethyl ether; ester solvents, e.g., ethyl acetate, butyl acetate, and isobutyl acetate; glycol ether ester solvents, e.g., ethylene glycol ethyl ether acetate, propylene glycol methyl ether acetate, 3-methyl-3-methoxybutyl acetate, and ethyl 3-ethoxypropionate; amide solvents, e.g., dimethylformamide (DMF), dimethylacetamido (DMAc), N-methyl-2-pyrrolidone (NMP), 1,3-dimethyl-2-imidazolidinone (DMI), and 1,3-dimethyl-3,4,5,6-tetrahydro-2(1H)-pyrimidinone (DMPU); and nitrile solvents, e.g., acetonitrile, propionitrile, butyronitrile, and benzonitrile. Of these, DMF, methyl ethyl ketone, ethyl acetate, acetone, and tetrahydrofuran are preferred in consideration of solvent recovery, and solubility, reactivity, boiling point, and emulsifying or dispersing ability in water during urethane synthesis.

The organic solvent is blended in an amount of up to 200 parts, preferably up to 160 parts by weight per 100 parts by weight of the silicone-modified polyurethane resin.

### (End Terminating Agent)

In the step of synthesizing the silicone-modified polyurethane resin used herein, when some isocyanate groups are left at polymer ends, termination reaction to the isocyanate end may further be performed. For example, not only monofunctional compounds such as monoalcohols and monoamines, but also compounds containing two functional groups having different reactivity to the isocyanate group may be used. Examples include monoalcohols such as methyl alcohol, ethyl alcohol, n-propyl alcohol, isopropyl alcohol, n-butyl alcohol, isobutyl alcohol, and t-butyl alcohol; monoamines such as monoethylamine, n-propylamine, diethylamine, di-n-propylamine, and di-n-butylamine; and alkanolamines such as monoethanolamine and diethnolamine. Of these, alkanolamines are preferred for ease of reaction control.

The silicone-modified polyurethane resin preferably has a number average molecular weight (Mn) of 10,000 to 200,000. As long as the Mn of the silicone-modified polyurethane resin falls within the range, polymer chains are fully entangled together in the polymer solution to facilitate fibrillization. Also from the standpoint of the polymer solution developing an adequate viscosity for fiber spinning by an electrospinning method, the Mn preferably falls within the above range. More preferably the Mn is 40,000 to 120,000.

### <Production Steps>

The nanofibers comprising the silicone-modified polyurethane resin is preferably produced through the following three steps. The first step is to produce the silicone-modified polyurethane resin, the second step is to prepare a solution or dispersion of the silicone-modified polyurethane resin using an organic solvent, water or a mixture thereof, and the third step is to spin fibers from the solution or dispersion of the silicone-modified polyurethane resin.

The first step of producing the silicone-modified polyurethane resin is, for example, by combining (A) the long chain polyol having a number average molecular weight of at least 500, (B) the short chain polyol having a number average molecular weight of less than 500, (C) the active hydrogen-containing organopolysiloxane, and (D) the polyisocyanate in such amounts that the equivalent ratio of isocyanate groups to active hydrogen groups may range from 0.9/1 to 1.1/1, and reacting them in the presence or absence of an organic solvent free of an active hydrogen group in the molecule typically at 20 to 150° C., preferably 50 to 110° C. in accordance with a one-shot process or multi-stage process. The resulting resin is subjected to a solvent removal step or solvent dilution step if necessary, thereby yielding the silicone-modified polyurethane resin (or emulsion thereof in water) used herein. The resulting solution or dispersion preferably has a solid concentration of 5 to 50% by weight, more preferably 10 to 40% by weight. The solid concentration within the range is preferred from the standpoints of stability and spinnability of the dispersion and cost. As used herein, the term “solid concentration” refers to a nonvolatile content after drying the solution or dispersion at 105° C. for 3 hours.

In the second step, a solution or dispersion of a resin containing the silicone-modified polyurethane resin is prepared using an organic solvent, water or mixture thereof.

The solvent used in the second step is not particularly limited as long as it has a boiling point of up to 300° C. at 1 atm., is liquid at 25° C., and dissolves the silicone-modified polyurethane resin and an optional resin. For example, the solvent used for polymerization of the silicone-modified polyurethane resin may be used, and the silicone-modified polyurethane resin solution obtained from polymerization may be used as such. Examples of the other solvent include organic solvents such as ether compounds, alcohol compounds, ketone compounds, amide compounds, nitrile compounds, aliphatic hydrocarbons, and aromatic hydrocarbons, typically dimethylformamide and methyl ethyl ketone, and water or mixtures thereof.

Examples of the ether compound include diethyl ether, t-butyl methyl ether (TBME), dibutyl ether, cyclopentyl methyl ether (CPME), diphenyl ether, dimethoxymethane (DMM), tetrahydrofuran (THF), 2-methyltetrahydrofuran, 2-ethyltetrahydrofuran, tetrahydropyran (THP), dioxane, trioxane, 1,2-dimethoxyethane, diethylene glycol dimethyl ether, and diethylene glycol diethyl ether, with THF being preferred.

Examples of the alcohol compound include methanol, ethanol, 1-propanol, 2-propanol, n-butyl alcohol, i-butyl alcohol, s-butyl alcohol, t-butyl alcohol, ethylene glycol, 2-methoxyethanol, 2-(2-methoxyethoxy)ethanol, 1,2-propanediol, 1,3-propanediol, 1,3-butanediol, 1,4-butanediol, 2,3-butanediol, 1,5-pentanediol, 2-butene-1,4-diol, 2-methyl-2,4-pentanediol, glycerol, 2-ethyl-2-mercaptomethyl-1,3-propanediol, 1,2,6-hexanetriol, cyclopentanol, cyclohexanol, and phenol, with methanol, ethanol, and ethylene glycol being preferred.

Examples of the ketone compound include methyl ethyl ketone, methyl isobutyl ketone, cyclopentanone, cyclohexanone, acetone, and limonene, with methyl ethyl ketone being preferred.

Examples of the amide compound include dimethylformamide (DMF), diethylformamide, dimethylacetamide (DMAc), N-methylpyrrolidone (NMP), N-ethylpyrrolidone, 1,3-dimethyl-2-imidazolidinone (DMI), and 1,3-dimethyl-3,4,5,6-tetrahydro-2(1H)-pyrimidinone (DMPU), with dimethylformamide being preferred.

Examples of the nitrile compound include acetonitrile, propionitrile, butyronitrile, and benzonitrile, with acetonitrile and propionitrile being preferred.

Examples of the aliphatic and aromatic hydrocarbons include toluene, o-xylene, m-xylene, p-xylene, 1,3,5-mesitylene, 1,2,3-mesitylene, 1,2,4-mesitylene, ethylbenzene, n-propylbenzene, i-propylbenzene, n-butylbenzene, i-butylbenzene, sec-butylbenzene, t-butylbenzene, n-pentylbenzene, i-pentylbenzene, sec-pentylbenzene, t-pentylbenzene, n-hexylbenzene, i-hexylbenzene, sec-hexylbenzene, t-hexylbenzene, cyclopentane, cyclohexane, cycloheptane, cyclooctane, methylcyclohexane, ethylcyclohexane, propylcyclohexane, n-butylcyclohexane, i-butylcyclohexane, sec-butylcyclohexane, t-butylcyclohexane, n-pentylcyclohexane, i-pentylcyclohexane, sec-pentylcyclohexane, t-pentylcyclohexane, n-hexylcyclohexane, i-hexylcyclohexane, sec-hexylcyclohexane, t-hexylcyclohexane, limonene, and α,α,α-trifluoromethylbenzene.

The solvent mixture is preferably a combination of an ether compound with water, an ether compound with an alcohol compound, a ketone compound with water, or an amide compound with a ketone compound. A solvent mixture of an amide compound and a ketone compound is more preferred. The mixing ratio of the amide compound to the ketone compound is preferably from 50:50 to 80:20 (both in weight ratio) because the use of a low-boiling ketone compound increases the evaporating rate and makes fiber spinning difficult.

The solution or dispersion of the resin containing the silicone-modified polyurethane resin preferably has a viscosity of 1 to 1,500 dPa·s, more preferably 200 to 800 dPa·s. Notably, the viscosity is measured at 25° C. by a rotational viscometer.

The temperature for preparation is not particularly limited. Since an increase of molecular weight brings about a lowering of solubility and a rise of viscosity, preparation may be conducted at elevated temperature. Stirring is desirably performed at a temperature in the range of, for example, room temperature to 80° C., preferably room temperature to 60° C. for 1 minute to 48 hours, preferably 1 hour to 24 hours as long as physical properties are not affected.

The third step is to spin fibers from the solution or dispersion of the silicone-modified polyurethane resin. Although the fiber spinning method is not particularly limited, electrospinning methods (e.g., electrostatic spinning, electrospinning, and melt spinning methods) are preferred.

### <Electrospinning Method>

In the electrospinning method, a non-woven fabric is obtained by injecting a polymer solution in an electrostatic field which is generated by applying a high voltage between electrodes on a nozzle and a collector, to form nanofibers and laying up the nanofibers on a collecting substrate. As used herein, the term “non-woven fabric” refers not only to one after evaporating and removing the solvent, but also to one containing the solvent.

Described below is a fiber spinning setup suitable for the electrospinning method preferably used in the invention. The electrodes used herein may be any electrodes of metal, inorganic or organic substances as long as they are electrically conductive. Also, insulating materials having a thin film of a conductive metal, inorganic or organic substance thereon may be used. The electrostatic field is established by applying a high voltage between a nozzle and a collector and may be established between a pair of or a multiplicity of electrodes. For example, the use of three electrodes, in total, consisting of two electrodes having different voltage values (e.g., 15 kV and 10 kV) and a grounded electrode and the use of more electrodes are encompassed.

In preparing nanofibers by the electrospinning method, the solvent may be used alone or in a combination of two or more. Examples of the method for controlling the evaporating rate of the solvent include adjusting the nozzle shape, using a solvent mixture, and adjusting the temperature or humidity in the fiber spinning environment, which may be used in a suitable combination. Of these, the method of using a solvent mixture is a simple and effective solution.

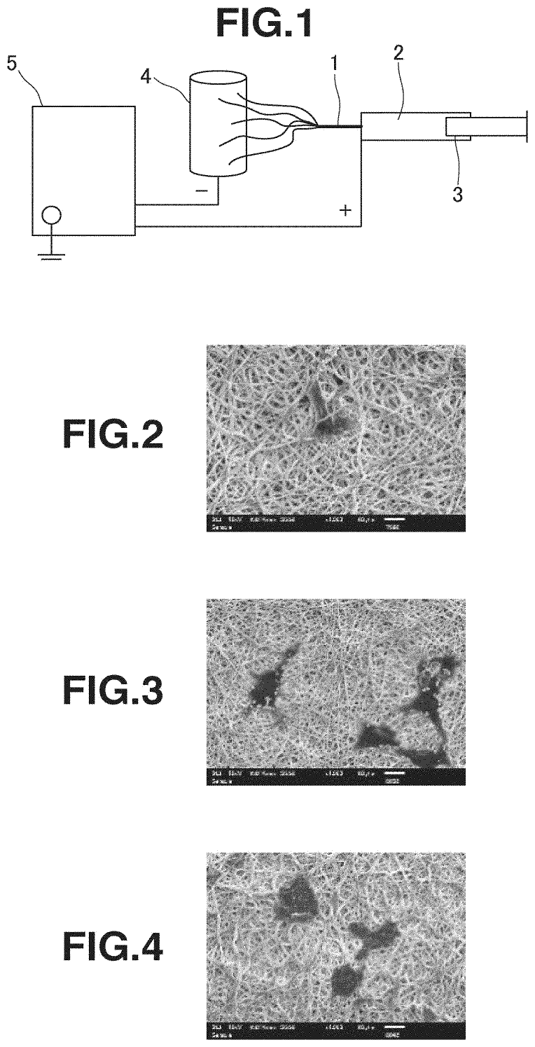

The polymer solution thus prepared may be discharged from a nozzle into the electrostatic field by any methods. For example, in FIG. 1, a polymer solution 2 is fed to a polymer solution reservoir equipped with a nozzle 1 and the polymer solution is ejected from the polymer reservoir nozzle set in the electrostatic field, to whip into fibers. Any suitable apparatus may be used for the process. For example, a syringe needle-shaped nozzle 1 is attached at the tip of a polymer solution-containing portion of a cylindrical syringe 3 and placed at an adequate distance from a collecting substrate 4 having a grounded electrode while a voltage is applied to the nozzle 1 by any suitable means such as a high-voltage supply 5. As the polymer solution 2 is ejected from the tip of the nozzle 1, it whips into nanofibers between the tip of the nozzle 1 and the collecting substrate 4.

In addition to the above method, well-known methods may be used for introducing the polymer solution into the electrostatic field. For example, an electrode which is paired with the electrode for collecting nanofibers may be directly inserted into a polymer solution-containing syringe having a nozzle. A tank may be used instead of the syringe because often the syringe is of a small volume. While applying pressure from the top of the tank, fiber spinning may be carried out from a nozzle located at the bottom of the tank. Inversely, while applying pressure from the bottom of the tank, fiber spinning may be carried out from a nozzle located at the top of the tank. In this case, an electrode may be placed near a blowing opening instead of directly attaching to the nozzle and assisting air may be used for laying up fibers on the collecting substrate (JP-A 2010-121221). As another fiber spinning method not using a nozzle, electrostatic spinning on a rotating roller is proposed. For example, the rotating roller is immersed in the bath filled with the polymer solution, the polymer solution is carried on the roller surface, and a high voltage is applied to the roller surface to perform electrostatic spinning.

When the polymer solution is fed from a nozzle into the electrostatic field, the production rate of a fiber layup structure may be increased by providing multiple nozzles (JP-A 2007-303031) or an assisting air blower (JP-A 2014-047440). For the purpose of improving the quality, nanofiber alignment may be improved by providing an electrode body between a nozzle and a collecting substrate to impart a preselected electric potential (JP-A 2008-223186); multiple nozzles may be provided with an assisting air blowing opening and the spacing between the nozzles be controlled (JP-A 2014-177728); and gear pumps may be used to deliver a mixed solution to multiple nozzles for ensuring a constant fiber diameter and accelerating a processing speed (JP-A 2010-189771). Although the distance between electrodes depends on the voltage, the nozzle size (i.e., diameter), the flow rate and concentration of the fiber spinning solution, and the like, for example, a distance of 5 to 30 cm is adequate at an applied voltage of 10 to 20 kV for preventing a corona discharge as well. Another method for preventing a corona discharge may be fiber spinning in vacuum.

The applied voltage is preferably 3 to 100 kV, more preferably 5 to 30 kV, though not limited thereto.

Although the size of the nozzle (inner diameter of the discharge opening) through which the polymer solution is ejected is not particularly limited, it is preferably 0.05 to 2 mm, more preferably 0.1 to 1 mm in consideration of a balance between productivity and the resulting fiber diameter.

The feed rate (or extrusion speed) of the polymer solution is not particularly limited. It is preferably determined as appropriate because it affects the target fiber diameter. For example, the feed rate of the polymer solution is preferably 0.01 to 0.1 ml/min per nozzle.

While the collecting substrate also serves as an electrode in the above configuration, nanofibers may be collected on a collecting substrate placed between electrodes. In this embodiment, continuous production becomes possible by placing a belt-shaped collecting substrate between electrodes.

When the polymer solution is deposited on the collecting substrate, the solvent evaporates and a fiber layup structure is formed. Generally, the solvent evaporates off at room temperature before capture on the collecting substrate. If the solvent evaporates insufficiently, fiber spinning may be performed under vacuum conditions. The temperature of the fiber spinning environment varies with a particular solvent and depends on the evaporation of the solvent and the viscosity of the polymer solution. The fiber spinning is typically performed at 0 to 50° C. When a low volatile solvent is used, a temperature of higher than 50° C. may be employed as long as the functions of the fiber spinning setup and the resulting fiber layup structure are not impaired. An adequate humidity is 0 to 50% RH, although it may be changed depending on the polymer concentration, the type of solvent, and the like. To this end, the syringe or tank for feeding the polymer solution may be equipped with a temperature control mechanism and a humidity control mechanism. As used herein, the term “polymer concentration” refers to a value computed by dividing the total weight of the polyurethane resin component in the polymer solution by the total weight of the polymer solution.

After the third step of spinning fibers from the solution or dispersion of the silicone-modified polyurethane resin, optional washing treatment and/or drying treatment may additionally be performed in order to remove the used organic solvents from the resulting fiber layup structure of nanofibers. Additionally, sterilization treatment may be performed in a certain application.

In washing treatment, liquids which dissolve the used organic solvent are preferably used. Water, deionized water, and distilled water are more preferred as viewed from the application. The temperature and time of drying treatment are not particularly limited. For example, by drying the resulting fiber layup structure of nanofibers at 80° C. for 24 hours, the organic solvent may be fully removed therefrom.

<Nanofibers Formed from a Resin Containing the Silicone-Modified Polyurethane Resin>

The nanofibers thus obtained have an average fiber diameter of less than 2,000 nm, preferably less than 1,000 nm, more preferably 400 to 700 nm. As long as the average fiber diameter falls within the range, the nanofibers exert good nano-size effects as medical material as compared with ordinary fibers.

The nanofibers may be used alone or in combination with another member, depending on ease of handling and other requirements. For example, a support base such as a non-woven fabric, woven fabric or film is used as the collecting substrate and the nanofibers are laid thereon. Then a composite material of the support base combined with the fiber layup structure can be prepared.

In conjunction with the fiber layup structure comprising nanofibers formed from a resin containing the silicone-modified polyurethane resin according to the invention, various additives such as inorganic or organic fillers and functional medicines may be blended in, adsorbed to or absorbed by the fiber layup structure, for the purpose of imparting or improving a variety of properties to the resulting nanofibers depending on a particular application, as long as the physical properties are not impaired.

The nanofibers used herein are formed from a resin containing the silicone-modified polyurethane resin. Although the resin preferably consists of the silicone-modified polyurethane resin, it may additionally contain one or more resins such as vinyl resins, acrylic resins, methacrylic resins, epoxy resins, urethane resins, olefin resins, and silicone resins in an amount of preferably 0 to 50% by weight, more preferably 0 to 20% by weight, if necessary.

As used herein, the term “fiber layup structure” refers to a three-dimensional structure which is formed by laying up, weaving, knitting or otherwise processing one or multiple nanofibers. Exemplary forms of the fiber layup structure include non-woven fabric, tube, and mesh.

The elastic modulus of the fiber layup structure of nanofibers is preferably 1 to 20 MPa, more preferably 2 to 10 MPa. The coefficient of dynamic friction on the surface is preferably 0.5 to 2.0, more preferably 0.5 to 1.0. The thermal conductivity is preferably 0.001 to 0.02 W/mK, more preferably 0.01 to 0.02 W/mK. The contact angle with water is preferably at least 100° (i.e., water repellent), more preferably 120 to 160°. The moisture regain is preferably up to 150%, more preferably 50 to 120%. The elongation at break is preferably at least 80%, more preferably at least 100%.

Notably, the elastic modulus, coefficient of dynamic friction, thermal conductivity, contact angle with water, and moisture regain are measured by the following methods.

### <Elastic Modulus>

The elastic modulus was determined from the stress-strain curve which was obtained by cutting the fiber layup structure into a test piece of 5 mm wide and 10 mm long and measuring at a pulling rate of 10 mm/min by means of a compact table-top material tester EZ Test/EZ-S(Shimadzu Corp.).

### <Coefficient of Dynamic Friction>

The coefficient of dynamic friction was determined by a horizontal tensile tester AGS-X (Shimadzu Corp.) under a load of 200 g and moving speed of 0.3 m/min.

Conditions: coefficient of dynamic friction between the fiber layup structure and woodfree paper.

### <Thermal Conductivity>

The thermal conductivity was measured by a precise and fast thermal property-measuring instrument KES-F7 Thermo Labo IIB (Kato Tech Co., Ltd.).

<Contact Angle with Water>

The static contact angle with pure water was measured by an automatic contact angle meter DM-501Hi (Kyowa Interface Science Co., Ltd.).

### <Moisture Regain>

The fiber layup structure was immersed in water for 24 hours and then dried under the conditions of JIS L1096 at 60° C. for 24 hours.

$$Moisture regain ( \% ) = [ ( weight ( g ) of sample before drying ) - ( weight ( g ) of dry sample ) ] / ( weight ( g ) of dry sample ) \times 100$$

In the invention, the fiber layup structure comprising the nanofibers is used as a material for medical use and medical instruments (i.e., medical material). Examples of the medical material include cell scaffold materials and culture substrates used in regenerative medical engineering, medical tubes such as catheters and artificial blood vessels, scratch-covering materials such as wound pads and gauze (i.e., wound dressing materials), and membrane filters for blood component separation. Especially, since the fiber layup structure comprising the nanofibers is excellent in cell adhesion and cell proliferation and has biocompatibility, it is advantageously used as cell scaffold materials and wound dressing materials.

### EXAMPLES

Examples and Comparative Examples are given below for illustrating the invention, but the invention is not limited thereto. In Examples and Comparative Examples, “parts” and “%” are by weight, unless otherwise stated. Evaluation in Examples and Comparative Examples was conducted by the following method.

In Examples, the number average molecular weight (Mn) was measured by gel permeation chromatography (GPC) versus polymethyl methacrylate (PMMA) standards. The GPC measurement was conducted under conditions including instrument: HLC-8320GPC (Tosoh Corp.), solvent (developing solvent): tetrahydrofuran (THF), and resin concentration: 0.1%. For infrared absorption spectroscopy (IR) analysis, NICOLET 6700 (Thermo Fisher Scientific Inc.) was used.

### <Synthesis of Silicone Block-Modified Urethane Resin>

### Synthesis Example 1-1: Synthesis of SiPU1-1

A reactor equipped with a stirrer, reflux condenser, thermometer, nitrogen inlet tube, and opening was furnished. While the reactor interior was purged with nitrogen gas, the reactor was charged with 200 g of poly(tetramethylene ether) glycol (trade name PolyTHF 1000 by BASF Japan, Ltd., Mn 1,000, hydroxyl number 113 mg KOH/g), 38 g of 1,4-butanediol, 45 g of a both end-type silicone diol (Compound (2-1), m=20), and 676.5 g of dimethylformamide (DMF). Heating and stirring was started and continued until the system became uniform, after which 168.0 g (the equivalent ratio of isocyanate groups to active hydrogen groups=1) of 4,4′-methylenebis(phenylene isocyanate) (MDI) was added thereto at 50° C. The temperature was raised at 80° C. for reaction. The reaction was conducted until the absorption peak at 2,270 cm⁻¹ assigned to a free isocyanate group on IR analysis disappeared. Thereafter, the reaction mixture was diluted with 60.1 g of DMF and 315.7 g of methyl ethyl ketone (MEK), yielding a solution of silicone polyurethane resin SiPU1-1 having a silicone content of 10.0%, a Mn of 73,000, and a solid concentration of 30%. The results are shown in Table 1.

### Synthesis Example 1-2: Synthesis of SiPU1-2

A reactor equipped with a stirrer, reflux condenser, thermometer, nitrogen inlet tube, and opening was furnished. While the reactor interior was purged with nitrogen gas, the reactor was charged with 155 g of poly(tetramethylene ether) glycol (trade name PolyTHF 1000 by BASF Japan, Ltd., Mn 1,000, hydroxyl number 113 mg KOH/g), 38 g of 1,4-butanediol, 90 g of a both end-type silicone diol (Compound (2-1), m=20), and 659.7 g of dimethylformamide (DMF). Heating and stirring was started and continued until the system became uniform, after which 156.8 g (the equivalent ratio of isocyanate groups to active hydrogen groups=1) of 4,4′-methylenebis(phenylene isocyanate) (MDI) was added thereto at 50° C. The temperature was raised at 80° C. for reaction. The reaction was conducted until the absorption peak at 2,270 cm⁻¹ assigned to a free isocyanate group on IR analysis disappeared. Thereafter, the reaction mixture was diluted with 58.6 g of DMF and 307.9 g of methyl ethyl ketone (MEK), yielding a solution of silicone polyurethane resin SiPU1-2 having a silicone content of 20.5%, a Mn of 88,000, and a solid concentration of 30%. The results are shown in Table 1.

### Synthesis Example 1-3: Synthesis of SiPU1-3

A reactor equipped with a stirrer, reflux condenser, thermometer, nitrogen inlet tube, and opening was furnished. While the reactor interior was purged with nitrogen gas, the reactor was charged with 75 g of poly(tetramethylene ether) glycol (trade name PolyTHF 1000 by BASF Japan, Ltd., Mn 1,000, hydroxyl number 113 mg KOH/g), 38 g of 1,4-butanediol, 170 g of a both end-type silicone diol (Compound (2-1), m=20), and 646.2 g of dimethylformamide (DMF). Heating and stirring was started and continued until the system became uniform, after which 147.8 g (the equivalent ratio of isocyanate groups to active hydrogen groups=1) of 4,4′-methylenebis(phenylene isocyanate) (MDI) was added thereto at 50° C. The temperature was raised at 80° C. for reaction. The reaction was conducted until the absorption peak at 2,270 cm⁻¹ assigned to a free isocyanate group on IR analysis disappeared. Thereafter, the reaction mixture was diluted with 57.4 g of DMF and 301.6 g of methyl ethyl ketone (MEK), yielding a solution of silicone polyurethane resin SiPU1-3 having a silicone content of 39.4%, a Mn of 102,000, and a solid concentration of 30%. The results are shown in Table 1.

### Synthesis Example 2: Synthesis of SiPU2

A reactor equipped with a stirrer, reflux condenser, thermometer, nitrogen inlet tube, and opening was furnished. While the reactor interior was purged with nitrogen gas, the reactor was charged with 200 g of poly(tetramethylene ether) glycol (trade name PolyTHF 1000 by BASF Japan, Ltd., Mn 1,000, hydroxyl number 113 mg KOH/g), 38 g of 1,4-butanediol, 45 g of a both end-type silicone diol (Compound (2-1), m=10), and 667.7 g of dimethylformamide (DMF). Heating and stirring was started and continued until the system became uniform, after which 162.1 g (the equivalent ratio of isocyanate groups to active hydrogen groups=1) of 4,4′-methylenebis(phenylene isocyanate) (MDI) was added thereto at 50° C. The temperature was raised at 80° C. for reaction. The reaction was conducted until the absorption peak at 2,270 cm⁻¹ assigned to a free isocyanate group on IR analysis disappeared. Thereafter, the reaction mixture was diluted with 60.3 g of DMF and 311.6 g of methyl ethyl ketone (MEK), yielding a solution of silicone polyurethane resin SiPU2 having a silicone content of 10.1%, a Mn of 87,000, and a solid concentration of 30%. The results are shown in Table 1.

### Synthesis Example 3: Synthesis of SiPU3

A reactor equipped with a stirrer, reflux condenser, thermometer, nitrogen inlet tube, and opening was furnished. While the reactor interior was purged with nitrogen gas, the reactor was charged with 200 g of poly(tetramethylene ether) glycol (trade name PolyTHF 1000 by BASF Japan, Ltd., Mn 1,000, hydroxyl number 113 mg KOH/g), 38 g of 1,4-butanediol, 45 g of a both end-type silicone diol (Compound (2-1), m=40), and 663.6 g of dimethylformamide (DMF). Heating and stirring was started and continued until the system became uniform, after which 159.4 g (the equivalent ratio of isocyanate groups to active hydrogen groups=1) of 4,4′-methylenebis(phenylene isocyanate) (MDI) was added thereto at 50° C. The temperature was raised at 80° C. for reaction. The reaction was conducted until the absorption peak at 2,270 cm⁻¹ assigned to a free isocyanate group on IR analysis disappeared. Thereafter, the reaction mixture was diluted with 59.0 g of DMF and 309.7 g of methyl ethyl ketone (MEK), yielding a solution of silicone polyurethane resin SiPU3 having a silicone content of 10.2%, a Mn of 79,000, and a solid concentration of 30%. The results are shown in Table 1.

### Synthesis Example 4: Synthesis of SiPU4

A reactor equipped with a stirrer, reflux condenser, thermometer, nitrogen inlet tube, and opening was furnished. While the reactor interior was purged with nitrogen gas, the reactor was charged with 200 g of poly(tetramethylene ether) glycol (trade name PolyTHF 1000 by BASF Japan, Ltd., Mn 1,000, hydroxyl number 113 mg KOH/g), 38 g of 1,4-butanediol, 45 g of a both end-type silicone diol (Compound (2-1), m=60), and 661.7 g of dimethylformamide (DMF). Heating and stirring was started and continued until the system became uniform, after which 158.1 g (the equivalent ratio of isocyanate groups to active hydrogen groups=1) of 4,4′-methylenebis(phenylene isocyanate) (MDI) was added thereto at 50° C. The temperature was raised at 80° C. for reaction. The reaction was conducted until the absorption peak at 2,270 cm⁻¹ assigned to a free isocyanate group on IR analysis disappeared. Thereafter, the reaction mixture was diluted with 58.8 g of DMF and 308.8 g of methyl ethyl ketone (MEK), yielding a solution of silicone polyurethane resin SiPU4 having a silicone content of 10.2%, a Mn of 75,000, and a solid concentration of 30%. The results are shown in Table 1.

### <Synthesis of Polyurethane Resin>

### Comparative Synthesis Example 1: Synthesis of PU1

A reactor equipped with a stirrer, reflux condenser, thermometer, nitrogen inlet tube, and opening was furnished. While the reactor interior was purged with nitrogen gas, the reactor was charged with 200 g of poly(tetramethylene ether) glycol (trade name PolyTHF 1000 by BASF Japan, Ltd., Mn 1,000, hydroxyl number 113 mg KOH/g), 38 g of 1,4-butanediol, and 590.9 g of dimethylformamide (DMF). Heating and stirring was started and continued until the system became uniform, after which 155.9 g (the equivalent ratio of isocyanate groups to active hydrogen groups=1) of 4,4′-methylenebis(phenylene isocyanate) (MDI) was added thereto at 50° C. The temperature was raised at 80° C. for reaction. The reaction was conducted until the absorption peak at 2,270 cm⁻¹ assigned to a free isocyanate group on IR analysis disappeared. Thereafter, the reaction mixture was diluted with 52.5 g of DMF and 275.7 g of methyl ethyl ketone (MEK), yielding a solution of silicone-free polyurethane resin PU1 having a silicone content of 0%, a Mn of 75,000, and a solid concentration of 30%. The results are shown in Table 1.

TABLE 1

| | | Synthesis | Synthesis | Synthesis | Synthesis | Synthesis | Synthesis | Comparative |
|---|---|---|---|---|---|---|---|---|
| | | Example | Example | Example | Example | Example | Example | Synthesis |
| | | 1-1 | 1-2 | 1-3 | 2 | 3 | 4 | Example 1 |
| Composition (g) | | SiPU1-1 | SiPU1-2 | SiPU1-3 | SiPU2 | SiPU3 | SiPU4 | PU1 |
| PolyTHF 1000 | | 200 | 155 | 75 | 200 | 200 | 200 | 200 |
| Compound (2-1) | m = 10 | — | — | — | 45 | — | — | — |
| | m = 20 | 45 | 90 | 170 | — | — | — | — |
| | m = 40 | — | — | — | — | 45 | — | — |
| | m = 60 | — | — | — | — | — | 45 | — |

| | | | | | | | |
|---|---|---|---|---|---|---|---|
| 1,4-Butanediol | 38 | 38 | 38 | 38 | 38 | 38 | 38 |
| MDI | 168.0 | 156.8 | 148.0 | 162.1 | 159.4 | 158.1 | 155.9 |
| DMF | 736.6 | 718.3 | 703.6 | 728.0 | 722.6 | 720.5 | 643.4 |
| MEK | 315.7 | 307.9 | 301.6 | 311.6 | 309.7 | 308.8 | 275.7 |
| Solid concentration (%) | 30 | 30 | 30 | 30 | 30 | 30 | 30 |
| Mn | 73,000 | 88,000 | 102,000 | 87,000 | 79,000 | 75,000 | 75,000 |
| Si content (%) | 10.0 | 20.5 | 39.5 | 10.1 | 10.2 | 10.2 | 0 |

### <Synthesis of Silicone Graft-Modified Polyurethane Resin>

### Synthesis Example 5: Synthesis of SiPU5

A reactor equipped with a stirrer, reflux condenser, thermometer, nitrogen inlet tube, and opening was furnished. While the reactor interior was purged with nitrogen gas, the reactor was charged with 200 g of poly(tetramethylene ether) glycol (trade name PolyTHF 1000 by BASF Japan, Ltd., Mn 1,000, hydroxyl number 113 mg KOH/g), 38 g of 1,4-butanediol, 50 g of a one end-type silicone diol (Compound (5-1), n=10), and 686.4 g of dimethylformamide (DMF). Heating and stirring was started and continued until the system became uniform, after which 169.6 g (the equivalent ratio of isocyanate groups to active hydrogen groups=1) of 4,4′-methylenebis(phenylene isocyanate) (MDI) was added thereto at 50° C. The temperature was raised at 80° C. for reaction. The reaction was conducted until the absorption peak at 2,270 cm⁻¹ assigned to a free isocyanate group on IR analysis disappeared. Thereafter, the reaction mixture was diluted with 61.0 g of DMF and 320.3 g of methyl ethyl ketone (MEK), yielding a solution of silicone polyurethane resin SiPU5 having a silicone content of 10.9%, a Mn of 71,000, and a solid concentration of 30%. The results are shown in Table 2.

### Synthesis Example 6: Synthesis of SiPU6

A reactor equipped with a stirrer, reflux condenser, thermometer, nitrogen inlet tube, and opening was furnished. While the reactor interior was purged with nitrogen gas, the reactor was charged with 200 g of poly(tetramethylene ether) glycol (trade name PolyTHF 1000 by BASF Japan, Ltd., Mn 1,000, hydroxyl number 113 mg KOH/g), 38 g of 1,4-butanediol, 48 g of a one end-type silicone diol (Compound (5-1), n=25), and 669.0 g of dimethylformamide (DMF). Heating and stirring was started and continued until the system became uniform, after which 160.6 g (the equivalent ratio of isocyanate groups to active hydrogen groups=1) of 4,4′-methylenebis(phenylene isocyanate) (MDI) was added thereto at 50° C. The temperature was raised at 80° C. for reaction. The reaction was conducted until the absorption peak at 2,270 cm⁻¹ assigned to a free isocyanate group on IR analysis disappeared. Thereafter, the reaction mixture was diluted with 59.5 g of DMF and 312.6 g of methyl ethyl ketone (MEK), yielding a solution of silicone polyurethane resin SiPU6 having a silicone content of 10.7%, a Mn of 70,000, and a solid concentration of 30%. The results are shown in Table 2.

### Synthesis Example 7: Synthesis of SiPU7

A reactor equipped with a stirrer, reflux condenser, thermometer, nitrogen inlet tube, and opening was furnished. While the reactor interior was purged with nitrogen gas, the reactor was charged with 200 g of poly(tetramethylene ether) glycol (trade name PolyTHF 1000 by BASF Japan, Ltd., Mn 1,000, hydroxyl number 113 mg KOH/g), 38 g of 1,4-butanediol, 48 g of a one end-type silicone diol (Compound (5-1), n=30), and 668.6 g of dimethylformamide (DMF). Heating and stirring was started and continued until the system became uniform, after which 159.7 g (the equivalent ratio of isocyanate groups to active hydrogen groups=1) of 4,4′-methylenebis(phenylene isocyanate) (MDI) was added thereto at 50° C. The temperature was raised at 80° C. for reaction. The reaction was conducted until the absorption peak at 2,270 cm⁻¹ assigned to a free isocyanate group on IR analysis disappeared. Thereafter, the reaction mixture was diluted with 59.4 g of DMF and 312.0 g of methyl ethyl ketone (MEK), yielding a solution of silicone polyurethane resin SiPU7 having a silicone content of 10.8%, a Mn of 72,000, and a solid concentration of 30%. The results are shown in Table 2.

### Synthesis Example 8: Synthesis of SiPU8

A reactor equipped with a stirrer, reflux condenser, thermometer, nitrogen inlet tube, and opening was furnished. While the reactor interior was purged with nitrogen gas, the reactor was charged with 200 g of poly(tetramethylene ether) glycol (trade name PolyTHF 1000 by BASF Japan, Ltd., Mn 1,000, hydroxyl number 113 mg KOH/g), 38 g of 1,4-butanediol, 47 g of a one end-type silicone diol (Compound (5-1), n=120), and 662.9 g of dimethylformamide (DMF). Heating and stirring was started and continued until the system became uniform, after which 156.9 g (the equivalent ratio of isocyanate groups to active hydrogen groups=1) of 4,4′-methylenebis(phenylene isocyanate) (MDI) was added thereto at 50° C. The temperature was raised at 80° C. for reaction. The reaction was conducted until the absorption peak at 2,270 cm⁻¹ assigned to a free isocyanate group on IR analysis disappeared. Thereafter, the reaction mixture was diluted with 58.9 g of DMF and 309.3 g of methyl ethyl ketone (MEK), yielding a solution of silicone polyurethane resin SiPU8 having a silicone content of 10.6%, a Mn of 78,000, and a solid concentration of 30%. The results are shown in Table 2.

TABLE 2

| | | Synthesis | Synthesis | Synthesis | Synthesis |
|---|---|---|---|---|---|
| | | Example | Example | Example | Example |
| | | 5 | 6 | 7 | 8 |
| Composition (g) | | SiPU5 | SiPU6 | SiPU7 | SiPU8 |
| PolyTHF 1000 | | 200 | 200 | 200 | 200 |
| Compound | n = 10 | 50 | — | — | — |
| (5-1) | n = 25 | — | 48 | — | — |
| | n = 30 | — | — | 48 | — |
| | n = 120 | — | — | — | 47 |

| | | | | |
|---|---|---|---|---|
| 1,4-Butanediol | 38 | 38 | 38 | 38 |
| MDI | 169.6 | 160.6 | 159.7 | 156.9 |
| DMF | 747.4 | 728.5 | 728.0 | 721.8 |
| MEK | 320.3 | 312.6 | 312.0 | 309.3 |
| Solid concentration (%) | 30 | 30 | 30 | 30 |
| Mn | 71,000 | 70,000 | 72,000 | 78,000 |
| Si content (%) | 10.9 | 10.7 | 10.8 | 10.6 |

### <Preparation of Fiber Layup Structure (Fiber Spinning Conditions)>

A solvent mixture of N,N-dimethylformamide and methyl ethyl ketone (weight ratio 64:36) was added to 3.0 g of the resin obtained in each Synthesis Example in the resin concentration shown in Table 3, which was stirred at room temperature for 24 hours, yielding a uniform milky white solution. Using the setup shown in FIG. 1, the polymer solution was discharged on the collecting substrate 4 for 10 hours under conditions including inner diameter of nozzle 1: 0.6 mm, distance from nozzle 1 to collecting substrate 4: 10 cm, and extrusion speed: 0.02 ml/min. The voltage applied during each fiber spinning is shown in Table 3.

On the scanning electron microscope (SEM) image of the resulting fiber layup structure, the fiber diameter was measured at 50 points, from which the average fiber diameter and the standard deviation (SD) were determined. The results are shown in Table 3.

TABLE 3

| | | | | | Average | |
|---|---|---|---|---|---|---|
| | | | | Applied | fiber | |
| Fiber layup | | | Resin conc. | voltage | diameter | SD |
| structure | Synthesis Example | Resin | (wt %) | (kV) | (nm) | (nm) |
| Structure 1-1 | Synthesis Example 1-1 | SiPU1-1 | 20 | 20 | 636 | 179 |
| Structure 1-2 | Synthesis Example 1-2 | SiPU1-2 | 13 | 17 | 531 | 142 |
| Structure 1-3 | Synthesis Example 1-3 | SiPU1-3 | 10 | 17 | 402 | 94 |
| Structure 2 | Synthesis Example 2 | SiPU2 | 20 | 20 | 690 | 150 |
| Structure 3 | Synthesis Example 3 | SiPU3 | 15 | 20 | 548 | 128 |
| Structure 4 | Synthesis Example 4 | SiPU4 | 13 | 20 | 440 | 89 |
| Structure 5 | Synthesis Example 5 | SiPU5 | 15 | 15 | 564 | 142 |
| Structure 6 | Synthesis Example 6 | SiPU6 | 15 | 15 | 544 | 124 |
| Structure 7 | Synthesis Example 7 | SiPU7 | 15 | 15 | 456 | 117 |
| Structure 8 | Synthesis Example 8 | SiPU8 | 15 | 15 | 456 | 129 |
| Comparative | Comparative Synthesis | PU1 | 15 | 20 | 720 | 215 |
| Structure 1 | Example 1 | | | | | |

### Example 1

The fiber layup structure thus obtained was evaluated for cell adhesion.

### <Pretreatments (Solvent Removal and Sterilization) of Specimen>

Before the evaluation of the fiber layup structure comprising the nanofibers, it was subjected to the following solvent removal as pretreatment in order to completely eliminate the impact of organic solvents therein. The resulting sample was stirred and washed in distilled water for 48 hours and dried at 80° C. for 24 hours.

After drying, the fiber layup structure comprising the nanofibers was subjected to the following sterilization as pretreatment for precise evaluation.

The resulting sample was cut into disks of diameter 10 mm, furnishing 3 specimens for each condition. Each of the specimens was set in each well of a 48-well culture plate (TPP tissue culture plate by TPP Techno Plastic Products AG), and secured by placing a glass cloning ring of outer diameter 9 mm (IWAKI, AGC Techno Glass Co., Ltd.) thereon. For sterilization, the specimens in the culture plate were immersed in 70% ethanol aqueous solution for 1 hour and washed 3 times with phosphate-buffered saline to remove the ethanol.

### <Cell Adhesion Assay>

NIH3T3 (mouse embryo fibroblast) was suspended in an Eagle's medium (about 50,000 cells per mL of the medium), added into the above sterilized wells of the culture plate, and incubated in an incubator at 37° C. for 3 hours. After incubation, the specimen was taken out of the culture plate and immersed in 1 mL of 0.5% Triton X-100/PBS (phosphate-buffered saline) solution for determination of the number of cells attached to the specimen, by the lactate dehydrogenase (LDH) method.

The LDH activity is determined by measuring the absorbance at a wavelength of 340 nm using a Thermo Scientific Multiskan FC microplate photometer (Thermo Fisher Scientific Inc.).

The enzyme activity of lactate dehydrogenase (LDH) may be measured from chemical reaction (i.e., amount) of LDH which is released from damaged or dead cells to the cell culture as a result of cell membrane injury. Using oxidized nicotinamide adenine dinucleotide (NAD) as a coenzyme, LDH converts lactate to form pyruvate and reduced nicotinamide adenine dinucleotide (NADH). Using the value of absorbance at 340 nm corresponding to the amount of the resulting NADH in the solution (i.e., the value which is proportional to the amount of LDH released into the cell culture from the specimen-attached cells by immersion in 0.5% Triton X-100/PBS solution), the number of cells present in the solution is determined as the number of adherent cells, based on the calibration curve previously depicted using known numbers of cells. The average value for 3 specimens is reported as an average number of adherent cells. The results are shown in Table 4.

TABLE 4

| | | | Average |
|---|---|---|---|
| | Fiber layup | | number of |
| Example | structure | Resin | adherent cells |
| Example 1-1 | Structure 1-1 | SiPU1-1 | 9,967 |
| Example 1-2 | Structure 1-3 | SiPU1-3 | 10,267 |
| Example 1-3 | Structure 3 | SiPU3 | 12,244 |
| Example 1-4 | Structure 4 | SiPU4 | 12,356 |
| Example 1-5 | Structure 5 | SiPU5 | 12,656 |
| Example 1-6 | Structure 6 | SiPU6 | 11,611 |
| Example 1-7 | Structure 7 | SiPU7 | 11,517 |
| Example 1-8 | Structure 8 | SiPU8 | 11,211 |
| Comparative | Comparative | PU1 | 9,256 |
| Example 1-1 | structure 1 | | |

### <Results of Cell Adhesion>

Each Example showed an increase of the number of adherent cells as compared with Comparative Example. This is probably because NIH3T3 (mouse embryo fibroblast) is likely to grow on the surface having a high water repellency due to silicone in the specimen, that is, hydrophobic surface.

When films of the resins in Synthesis Examples were prepared and similarly evaluated, the adhesion of cells was not observed.

### Example 2

After the fiber layup structure obtained above was subjected to pretreatments (solvent removal and sterilization) as in Example 1, it was evaluated for cell proliferation.

### <Cell Proliferation Assay>

NIH3T3 (mouse embryo fibroblast) was suspended in an Eagle's medium (about 50,000 cells per mL of the medium), added into the above sterilized wells of the culture plate, and incubated in an incubator at 37° C. for certain periods (1 day and 3 days). After incubation, the specimen was taken out of the culture plate and immersed in 1 mL of 0.5% Triton X-100/PBS solution for determination of the number of cells proliferated on the specimen surface, by the LDH method.

For the evaluation of the number of proliferated cells by the LDH method, the numbers of cells on the specimens after 1 day and 3 days of incubation were determined by the same procedure as in Example 1. A difference from the average number of adherent cells determined as in Example 1 was reported as an average number of proliferated cells. The results are shown in Table 5.

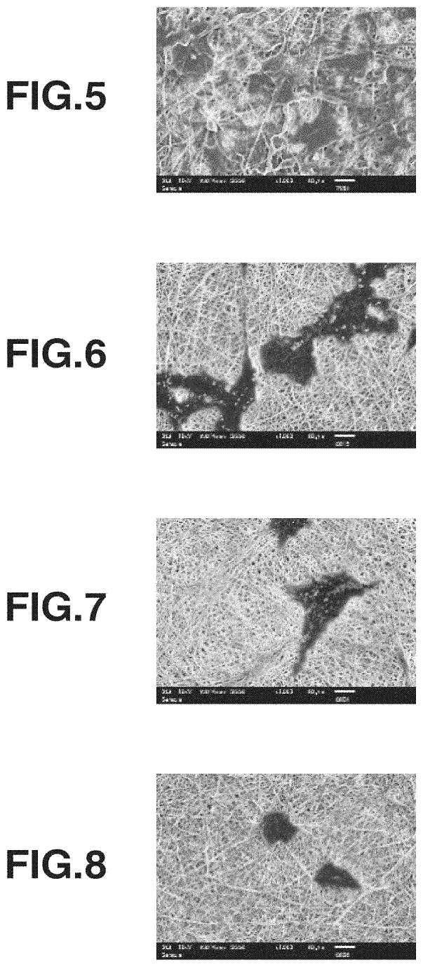

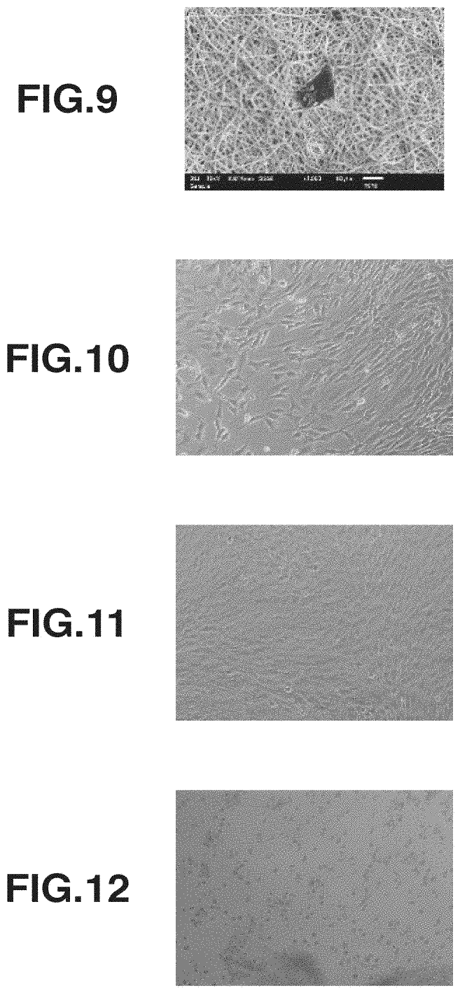

TABLE 5

| | | | Average number | | |
|---|---|---|---|---|---|
| | | | of adherent | | SEM |
| | | | (cells) | | observation |
| | Fiber layup | | 1-day | 3-day | 3-day |
| Example | structure | Resin | incubation | incubation | incubation |
| Example 2-1 | Structure 1-1 | SiPU1-1 | 6,238 | 3,411 | FIG. 2 |
| Example 2-2 | Structure 1-2 | SiPU1-2 | 3,625 | 8,741 | FIG. 3 |
| Example 2-3 | Structure 1-3 | SiPU1-3 | 2,946 | 8,926 | FIG. 4 |
| Example 2-4 | Structure 4 | SiPU4 | 628 | 5,500 | FIG. 5 |
| Example 2-5 | Structure 5 | SiPU5 | 2,570 | 7,970 | FIG. 6 |
| Example 2-6 | Structure 7 | SiPU7 | 2,622 | 6,656 | FIG. 7 |
| Example 2-7 | Structure 8 | SiPU8 | 3,081 | 9,611 | FIG. 8 |
| Comparative | Comparative | PU1 | 2,350 | 3,178 | FIG. 9 |
| Example 2-1 | structure 1 | | | | |

Separately, cell proliferation on the specimen surface after 3 days of incubation was examined. The specimen surface and cell shape were observed under a scanning electron microscope (SEM), JSM-6010LA by JEOL Ltd. (fiber layup structure: magnification 1,000×, acceleration voltage 10 kV). After 3 days of incubation, the medium was removed from the plate to stop proliferation of cells. For retaining the cell shape, the cells were fixed by adding a paraformaldehyde (PFA) solution as a crosslinker to the plate. The specimens were dehydrated by a sequential process involving 30 minutes of immersion in each of the ethanol gradient solutions (50, 70, 95, and 99.5%), followed by drying at room temperature. The specimens were coated with platinum for SEM observation.

The SEM images (1,000×) of the specimens in Examples after 3 days of incubation are shown in FIGS. 2 to 9.

### <Results of Cell Proliferation>

Each Example had a more average number of proliferated cells after 3 days of incubation than Comparative Example. As seen from the SEM images, the cells in Examples 2-1 to 2-7 had shapes proliferated in conformity with the shape of nanofibers, indicating that the cells proliferated using the nanofibers as a scaffold. It is proven that the fiber layup structure is a material suited for cell proliferation.

The results of Examples demonstrate that the fiber layup structure comprising nanofibers is advantageously used as a scaffold material for tissue engineering because its characteristics reside in a structure suitable for mimicking the extracellular matrix (ECM) and also a structure capable of increasing the stability of cell migration.

### Example 3

After the fiber layup structures obtained above (10 types: structures 1-1, 1-2, 1-3, 2 to 8) were subjected to pretreatments (solvent removal and sterilization) as in Example 1, they were evaluated for cytotoxicity as follows. Notably, the specimen was a disk having a diameter of about 10 mm and sterilized without placing in a culture plate. A medical nitrile glove was cut into a disk of diameter 10 mm, which was used as positive control material.

### <Method of Cytotoxicity Evaluation>

For the qualitative evaluation of biocompatibility, the fiber layup structure comprising nanofibers was evaluated for cytotoxicity as follows using the direct contact test of cells according to ISO 10993-5.

NIH3T3 mouse embryonic fibroblasts were established by collecting from a NIH Swiss mouse, transferring every 3 days and inoculating into a 50-mm dish at a density of 3×10⁵ (300,000) cells per 50-mm dish. The cells were inoculated on the surface of each plate, and incubated at 37° C. until the entire surface of the plate was covered with cells. Next the fiber layup structure specimen was placed on the cell layer at the center of the plate and secured by placing a cloning ring (IWAKI, AGC Techno Glass Co., Ltd.) thereon whereby the medium was changed. After 24 hours of incubation, the specimen was removed from each plate, to which trypan blue was added. The morphology change was observed under a microscope (40×). The toxicity was rated according to criteria: from Grade 0 (no toxicity) to Grade 4 (severe toxicity).

### <Results of Cytotoxicity Evaluation>

In the evaluation under the above conditions, structures 1-1, 1-2, 1-3, 2 to 8 were all rated Grade 0. That is, they did not exhibit toxicity upon contact with cells.

The results of microscopic observation of structure 4 (SiPU4) and structure 8 (SiPU8) are also shown in FIGS. 10 and 11, respectively. As compared with the case of the positive control material shown in FIG. 12, the cells maintained their shapes and remained viable.

As seen from these results, the nanofibers of the silicone-modified polyurethane resin in the specimen do not adversely affect the cytotoxicity or biocompatibility of the material.

These results demonstrate that the nanofibers of the silicone-modified polyurethane resin provide better cell adhesion and cell proliferation than non-silicone-modified polyurethane resins and have biocompatibility suitable as biomedical materials such as scaffold materials for tissue engineering (e.g., cell scaffold materials) and wound dressing materials (e.g., waterproof bandages).

### INDUSTRIAL APPLICABILITY

Since the medical material comprising nanofibers formed from a resin containing the silicone-modified polyurethane resin defined above has better cell adhesion and cell proliferation than non-silicone-modified polyurethane resins, it can be used as, for example, cell scaffold materials, artificial skins, artificial organs, and wound dressing materials such as wound bandages. The invention contributes to medical and medical instrument fields.

### REFERENCE SIGNS LIST

1 Nozzle
2 Polymer solution
3 Syringe (polymer solution reservoir)
4 Collecting substrate
5 High-voltage supply

1. A medical material comprising nanofibers formed from a resin containing a silicone-modified polyurethane resin, the silicone-modified polyurethane resin comprising the reaction product of (A) a long chain polyol having a number average molecular weight of at least 500, (B) a short chain polyol having a number average molecular weight of less than 500, (C) an active hydrogen-containing organopolysiloxane, and (D) a polyisocyanate, the nanofibers having an average fiber diameter of less than 2,000 nm.

2. The medical material of claim 1 wherein component (C) is (C-1) an organopolysiloxane represented by the following formula (1) and/or (C-2) an organopolysiloxane having carbinol groups only at one end of the molecular chain, represented by the following formula (2):
R¹R²₂SiO(SiR²₂O)ₘSiR¹R²₂ (1)
wherein R¹ is each independently a C₁-C₁₀ monovalent hydrocarbon group which has a hydroxy or mercapto group and may have an oxygen atom intervening in the chain, or a C₁-C₁₀ monovalent hydrocarbon group having a primary or secondary amino group, R² is each independently a straight, branched or cyclic C₁-C₁₀ alkyl or aralkyl group in which some hydrogen may be substituted by fluorine, optionally substituted C₅-C₁₂ aryl group, or vinyl group, and m is an integer of 1 to 200,
R²₃SiO(SiR²₂O)ₙSiR²₂R⁴ (2)
wherein R² is as defined above, R⁴ is a characteristic group having the following formula (3):
—R⁵—X—CH₂C(R⁶)₂R⁷ (3)
wherein R⁵ is a C₂-C₁₀ alkylene group which may contain an oxygen atom in the chain, R⁶ is a C₁-C₁₀ carbinol group, R⁷ is hydrogen, an amino group or C₁-C₁₀ alkyl group, and X is a single bond or —O— bond, with the proviso that when R⁵ contains an oxygen atom and X is a —O— bond, these two oxygen atoms do not adjoin each other, and n is an integer of 1 to 200.

3. The medical material of claim 1 wherein the average fiber diameter is less than 1,000 nm.

4. The medical material of claim 1 wherein component (C) is blended in an amount of 0.1 to 50 parts by weight per 100 parts by weight of components (A) to (D) combined.

5. The medical material of claim 1, which is in the form of a fiber layup structure comprising the nanofibers.

6. The medical material of claim 1, which is a cell scaffold material.

7. The medical material of claim 1, which is a wound dressing material.

8. A method for preparing the nanofiber-containing medical material of claim 1, comprising the step of spinning the nanofibers from a solution or dispersion of the silicone-modified polyurethane resin by an electrospinning method.

9. The method of claim 8, further comprising the step of washing and/or drying the nanofibers spun by an electrospinning method.

10. The method of claim 9, further comprising the step of sterilizing the washed and/or dried nanofibers.
